SEQUENCE LISTING (18 sequences)

SEQ ID NO: 1 - DNA, 15 bp, Artificial Sequence
  Description of Artificial Sequence Synthetic primer
tacggraggcagcag

SEQ ID NO: 2 - DNA, 18 bp, Artificial Sequence
  Description of Artificial Sequence Synthetic primer
acracacgagctgacgac

SEQ ID NO: 3 - DNA, 120 bp, Acidaminococcus intestini
actcaatgtcaagccttggtaaggttcttcgcgttgcgtcgaattaaaccacatactccaccgcttgtgcgggcccccgtcaattcctttgagtttcaatcttgcgatcgtagtccccag

SEQ ID NO: 4 - protein, 40 aa, Acidaminococcus intestini
TQCQALVRFFALRRIKPHTPPLVRAPVNSFEFQSCDRSPQ

SEQ ID NO: 5 - DNA, 121 bp, Acidaminococcus intestini
actcaatgtcaagccttggtaaggttcttcgcgttgcgtcgaattaaaccacatactccaccgcttgtgcgggcccccgtcaattcctttgagtttcaatcttgcgatcgtagtccccagg

SEQ ID NO: 6 - DNA, 122 bp, Acidaminococcus intestini
actcaatgtcaagccttggtaaggttcttcgcgttgcgtcgaattaaaccacatactccaccgcttgtgcgggcccccgtcaattcctttgagtttcaatcttgcgatcgtagtccccaggc

SEQ ID NO: 7 - protein, 15 aa, Acidaminococcus intestini
HSMSSLGKVLRVASN

SEQ ID NO: 8 - protein, 14 aa, Acidaminococcus intestini
TTYSTACAGPRQFL

SEQ ID NO: 9 - protein, 6 aa, Acidaminococcus intestini
VSILRS

SEQ ID NO: 10 - protein, 6 aa, Acidaminococcus intestini
LNVKPW

SEQ ID NO: 11 - protein, 33 aa, Acidaminococcus intestini
GSSRCVELNHILHRLCGPPSIPLSFNLAIVVPR

SEQ ID NO: 12 - DNA, 157 bp, Unknown
  Description of Unknown 16s rRNA sequence
  modified_base (8)..(11): a, c, t, g, unknown or other
  modified_base (16)..(16): a, c, t, g, unknown or other
  modified_base (26)..(27): a, c, t, g, unknown or other
  modified_base (30)..(31): a, c, t, g, unknown or other
  modified_base (38)..(38): a, c, t, g, unknown or other
  modified_base (72)..(72): a, c, t, g, unknown or other
  modified_base (85)..(85): a, c, t, g, unknown or other
  modified_base (90)..(90): a, c, t, g, unknown or other
  modified_base (101)..(101): a, c, t, g, unknown or other
  modified_base (115)..(116): a, c, t, g, unknown or other
  modified_base (124)..(124): a, c, t, g, unknown or other
  modified_base (126)..(126): a, c, t, g, unknown or other
  modified_base (134)..(134): a, c, t, g, unknown or other
  modified_base (136)..(136): a, c, t, g, unknown or other
  modified_base (144)..(144): a, c, t, g, unknown or other
  modified_base (157)..(157): a, c, t, g, unknown or other
aggttctnnnngttgnttcaaattannccnnatgctcnaccgcttgtgcgggtccccgtcaattcctttgantttcattcttgcnaacgnactccccaggnggaatacttattgnntttgctgnancaccgaanancttatganccccgacacctan

SEQ ID NO: 13 - protein, 52 aa, Unknown
  Description of Unknown 16s rRNA sequence
  MOD_RES (3)..(3): Any amino acid
  MOD_RES (4)..(4): C, R, S or G
  MOD_RES (5)..(5): C or W
  MOD_RES (9)..(9): F, L, I, V, S, P, T, A, Y, H, N, D, C, R, P or G
  MOD_RES (10)..(10): L, P, H, Q or R
  MOD_RES (13)..(13): Y, H, N or D
  MOD_RES (24)..(24): I, T, N or S
  MOD_RES (30)..(30): V, A, E or G
  MOD_RES (34)..(34): W, R or G
  MOD_RES (38)..(38): C or W
  MOD_RES (39)..(39): F, L, I, V
  MOD_RES (41)..(41): C or W
  MOD_RES (42)..(42): I, T, N or S
  MOD_RES (45)..(45): Y, H, Q, N, K, D or E
  MOD_RES (48)..(48): I, T, N or S
GSXXXFKLXXMLXRLCGSPSIPLXFILANXLPRXNTYXXCXXTEXLMXPDTY

SEQ ID NO: 14 - DNA, 162 bp, Akkermansia sp.
actccaccgcttgtgcgggtccccgtcaatttctttgagttttaatcttgcgaccgtactccccaggcggcacgcttaacgcgttagctccggcacgcagggggtcgattccccgcacaccaagcgtgcaccgtttactgccaggactacaggggtatctaa

SEQ ID NO: 15 - protein, 12 aa, Akkermansia sp.
YSTACAGPRQFL

SEQ ID NO: 16 - protein, 41 aa, Akkermansia sp.
VLILRPYSPGGTLNALAPARRGSIPRTPSVHRLLPGLQGYL

SEQ ID NO: 17 - DNA, 163 bp, Akkermansia sp.
actccaccgcttgtgcgggtccccgtcaatttctttgagttttaatcttgcgaccgtactccccaggcggcacgcttaacgcgttagctccggcacgcagggggtcgattccccgcacaccaagcgtgcaccgtttactgccaggactacaggggtatctaat

SEQ ID NO: 18 - protein, 42 aa, Akkermansia sp.
VLILRPYSPGGTLNALAPARRGSIPRTPSVHRLLPGLQGYLI

(19) United States
(12) Patent Application Publication
(10) Pub. No.: US 2021/0310051 A1
(43) Pub. Date: Oct. 7, 2021

(54) SYSTEMS AND METHODS FOR CHARACTERIZING COMPOSITIONS COMPRISING FECAL-DERIVED BACTERIAL POPULATIONS

(71) Applicant: Nubiyota, LLC, Teaneck, NJ (US)

(72) Inventors: Emma Allen-Vercoe, Teaneck, NJ (US); Shawn Langer, Teaneck, NJ (US); Nissim Mashiach, Teaneck, NJ (US)

(21) Appl. No.: 16/076,259

(22) PCT Filed: Feb. 10, 2017
(86) PCT No.: PCT/IB2017/000205

§ 371 Date: Aug. 7, 2018

Related U.S. Application Data
(60) Provisional application No. 62/294,125, filed on Feb. 11, 2016.

(51) Int. Cl. C12Q 1/689; A61K 35/74; C12N 1/20

(52) U.S. Cl. CPC C12Q 1/689; C12N 1/20; A61K 35/74

(57) ABSTRACT

In one embodiment, the present invention provides a method for characterizing a first composition comprising a fecal-derived bacterial population, comprising the steps of: obtaining a live culture of the first composition comprising a fecal-derived bacterial population, supplying factors that selectively expand the at least one bacterial strain at a level below a threshold for detection above the threshold level by seeding a chemostat containing culture medium with a second composition comprising a fecal-derived bacterial population; adding the live culture of the first composition comprising a fecal-derived bacterial population to the seeded chemostat, and culturing the first composition comprising a fecal-derived bacterial population with the second composition comprising a fecal-derived bacterial population in the chemostat for a time sufficient to expand the at least one bacterial strain; removing a sample of the chemostat culture; and identifying the at least one bacterial strain.

### RELATED APPLICATIONS

This application claims the priority of U.S. Patent Appln. No. 62/294,125; filed Feb. 11, 2016; entitled “SYSTEMS AND METHODS FOR CHARACTERIZING COMPOSITIONS COMPRISING FECAL-DERIVED BACTERIAL POPULATIONS,” which is incorporated herein by reference in its entirety for all purposes.

### FIELD OF THE INVENTION

The field of invention relates to therapies for treating gastrointestinal disorders. In particular, the present invention provides systems and methods for characterizing compositions comprising fecal-derived bacterial populations used as therapies for treating gastrointestinal disorders.

### BACKGROUND OF THE INVENTION

Compositions comprising fecal-derived bacterial populations may be used to treat gastrointestinal disorders.

### BRIEF DESCRIPTION OF THE FIGURES

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIGS. 1A and 1B show a single-stage chemostat vessel employed in the methods according to some embodiments of the present invention.

FIG. 2 shows a metabolic profile according to one embodiment of the present invention.

FIG. 3 shows a metabolic profile according to one embodiment of the present invention.

FIG. 4 shows sequence data obtained 16S rRNA profiling via the Sanger sequencing method.

FIG. 5 shows sequence data obtained 16S rRNA profiling via the Sanger sequencing method.

FIG. 6 shows sequence data obtained 16S rRNA profiling via the Sanger sequencing method, of at least one bacterial strain below a threshold level for detection, cultured to a level detectable by Sanger sequencing, by a method according to some embodiments of the present invention.

FIG. 7 shows an agarose gel of results of PCR reactions of MET-1+ samples, using primers specific for Akkermansia.

### SUMMARY OF THE INVENTION

In one embodiment, the present invention provides a method for characterizing a first composition comprising a fecal-derived bacterial population, comprising the steps of:
a. obtaining a live culture of the first composition comprising a fecal-derived bacterial population, wherein the first composition comprising a fecal-derived bacterial population comprises at least one bacterial strain at a level below a threshold for detection;
b. supplying factors that selectively expand the at least one bacterial strain at a level below a threshold for detection above the threshold level for detection by seeding a chemostat containing culture medium with a second composition comprising a fecal-derived bacterial population;
c. adding the live culture of the first composition comprising a fecal-derived bacterial population to the seeded chemostat, and culturing the first composition comprising a fecal-derived bacterial population with the second composition comprising a fecal-derived bacterial population in the chemostat for a time sufficient to expand the at least one bacterial strain at a level below a threshold for detection above the threshold level for detection;
d. removing a sample of the chemostat culture after the sufficient time; and
e. identifying the at least one bacterial strain at a level below a threshold for detection.

In one embodiment, the second composition comprising a fecal-derived bacterial population may also comprise at least one bacterial strain at a level below a threshold for detection.

In one embodiment, the method further comprises determining if the identified at least one bacterial strain at a level below a threshold for detection is from the first composition comprising a fecal-derived bacterial population, or the second composition comprising a fecal-derived bacterial population.

In one embodiment, the at least one bacterial strain at a level below a threshold for detection is identified by comparing a bacterial 16S rRNA profile of the first composition comprising a fecal-derived bacterial population obtained prior to culture in the chemostat, to a bacterial 16S rRNA of the chemostat culture medium, obtained after culture for a sufficient time.

In one embodiment, the first composition comprising a fecal-derived bacterial population comprises an ecosystem of a healthy patient.

In one embodiment, the second composition comprising a fecal-derived bacterial population comprises an ecosystem of a healthy patient.

In one embodiment, the first and the second composition comprising a fecal-derived bacterial population are the same.

In one embodiment, the first composition comprising a fecal-derived bacterial population is derived from a patient with a gut dysbiosis.

In one embodiment, the first composition comprising a fecal-derived bacterial population is derived from a patient with a gastrointestinal disease.

In one embodiment, the second composition comprising a fecal-derived bacterial population is derived from a patient with a gut dysbiosis.

In one embodiment, the second composition comprising a fecal-derived bacterial population is derived from a patient with a gastrointestinal disease.

In one embodiment, the gastrointestinal disease is selected from the group consisting of: dysbiosis, Clostridium difficile (Clostridioides difficile) infection, Crohn's disease, ulcerative colitis, irritable bowel syndrome, inflammatory bowel disease and diverticular disease.

In some embodiments, determining if the newly identified bacterial strains are from the first composition comprising a fecal-derived bacterial population, or the second composition comprising a fecal-derived bacterial population is performed via PCR on a sample of either the first or second culture, using primers specific for the newly identified bacterial strain.

### DETAILED DESCRIPTION OF THE INVENTION

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the term “OTU” refers to an operational taxonomic unit, defining a species, or a group of species via similarities in nucleic acid sequences, including, but not limited to 16S rRNA gene sequences.

In some embodiments, the present invention provides a method for characterizing a first composition comprising a fecal-derived bacterial population, comprising the steps of:
a. obtaining a live culture of the first composition comprising a fecal-derived bacterial population, wherein the first composition comprising a fecal-derived bacterial population comprises at least one bacterial strain at a level below a threshold for detection;
b. supplying factors that selectively expand the at least one bacterial strain at a level below a threshold for detection above the threshold level for detection by seeding a chemostat containing culture medium with a second composition comprising a fecal-derived bacterial population;
c. adding the live culture of the first composition comprising a fecal-derived bacterial population to the seeded chemostat, and culturing the first composition comprising a fecal-derived bacterial population with the second composition comprising a fecal-derived bacterial population in the chemostat for a time sufficient to expand the at least one bacterial strain at a level below a threshold for detection above the threshold level for detection;
d. removing a sample of the chemostat culture after the sufficient time; and
e. identifying the at least one bacterial strain at a level below a threshold for detection.

In some embodiments, the second composition comprising a fecal-derived bacterial population may also comprise at least one bacterial strain at a level below a threshold for detection.

### Fecal-Derived Bacterial Populations

In some embodiments, the first composition comprising a fecal-derived bacterial population comprises an ecosystem of a healthy patient.

In some embodiments, the second composition comprising a fecal-derived bacterial population comprises an ecosystem of a healthy patient.

In some embodiments, the first and the second composition comprising a fecal-derived bacterial population are the same.

In some embodiments, the first composition comprising a fecal-derived bacterial population is a bacterial composition disclosed in U.S. Patent Application Publication No. 20150044173. Alternatively, in some embodiments, the first composition comprising a fecal-derived bacterial population is a bacterial composition disclosed in U.S. Patent Application Publication No. 20140363397. Alternatively, in some embodiments, the first composition comprising a fecal-derived bacterial population is a bacterial composition disclosed in U.S. Patent Application Publication No. 20140086877. Alternatively, in some embodiments, the first composition comprising a fecal-derived bacterial population is a bacterial composition disclosed in U.S. Pat. No. 8,906,668.

In some embodiments, the second composition comprising a fecal-derived bacterial population is a bacterial composition disclosed in U.S. Patent Application Publication No. 20150044173. Alternatively, in some embodiments, the second composition comprising a fecal-derived bacterial population is a bacterial composition disclosed in U.S. Patent Application Publication No. 20140363397. Alternatively, in some embodiments, the second composition comprising a fecal-derived bacterial population is a bacterial composition disclosed in U.S. Patent Application Publication No. 20140086877. Alternatively, in some embodiments, the second composition comprising a fecal-derived bacterial population is a bacterial composition disclosed in U.S. Pat. No. 8,906,668.

In some embodiments, the first composition comprising a fecal-derived bacterial population is derived from a patient with a gastrointestinal disease. In some embodiments, the first composition comprising a fecal-derived bacterial population is derived from a patient with a gastrointestinal disease according to the methods disclosed in U.S. Patent Application Publication No. 20140342438.

In some embodiments, the second composition comprising a fecal-derived bacterial population is derived from a patient with a gastrointestinal disease. In some embodiments, the second composition comprising a fecal-derived bacterial population is derived from a patient with a gastrointestinal disease according to the methods disclosed in U.S. Patent Application Publication No. 20140342438.

In some embodiments, the gastrointestinal disease is selected from the group consisting of: dysbiosis, Clostridium difficile infection, Crohn's disease, ulcerative colitis, irritable bowel syndrome, inflammatory bowel disease and diverticular disease.

In some embodiments, the second composition comprising a fecal-derived bacterial population is derived from a patient by a method comprising:
a. obtaining a freshly voided stool sample, and placing the sample in an anaerobic chamber (in an atmosphere of 90% N2, 5% CO2 and 5% H2);
b. generating a fecal slurry by macerating the stool sample in a buffer; and
c. removing food particles by centrifugation, and retaining the supernatant.

In some embodiments, the supernatant is used to seed the chemostat.

### Culture Methods According to Some Embodiments of the Present Invention

The effectiveness of method to characterize bacterial populations can be limited by factors such as, for example, the sensitivity of the method (i.e. the method is only capable of detecting a particular bacterial strain if the strain is present above a threshold level).

In some embodiments, the threshold level is dependent on the sensitivity of the detection method. Thus, in some embodiments, depending on the sensitivity of the detection method, a greater amount of sample material is required to detect the least one bacterial strain at a level below a threshold for detection. In some embodiments, the greater amount of starting material is obtained by culturing the first composition comprising a fecal-derived bacterial population with the second composition comprising a fecal-derived bacterial population in the chemostat for a greater period of time.

In some embodiments, a first composition comprising a fecal-derived bacterial population comprises at least one bacterial strain at a level below a threshold for detection. In some embodiments, the first composition comprising a fecal-derived bacterial population is cultured with a second composition comprising a fecal-derived bacterial population.

Without intending to be limited to a particular theory, the at least one bacterial strain at a level below a threshold for detection may be refractory to culture in vitro, and the second composition comprising a fecal-derived bacterial population provides growth factors, supplements, metabolites, and any combination thereof, to enable the at least one bacterial strain at a level below a threshold for detection to grow in vitro. In some embodiments, the methods of the present invention culture the at least one bacterial strain at a level below a threshold for detection to a level above the threshold of detection, thereby enabling the at least one bacterial strain at a level below a threshold for detection to be detected and identified.

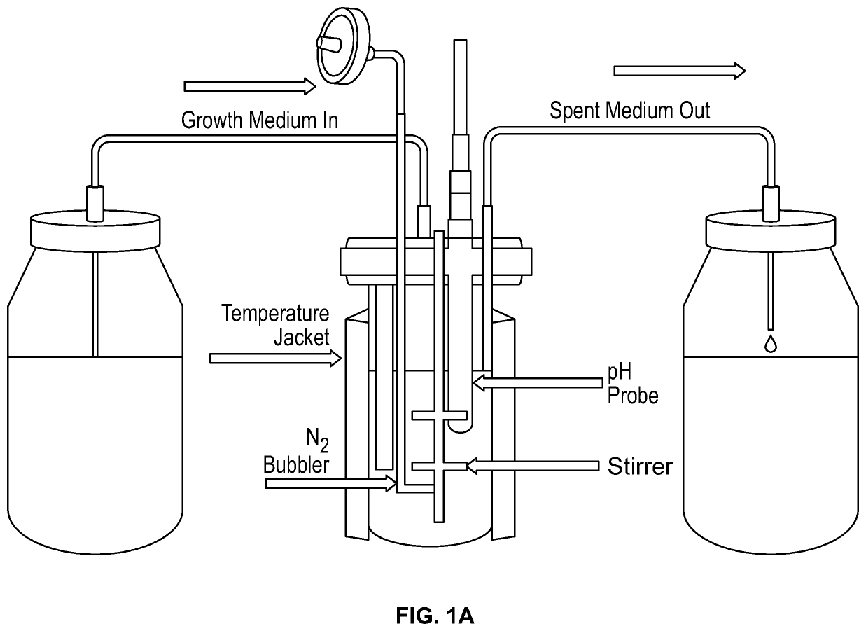

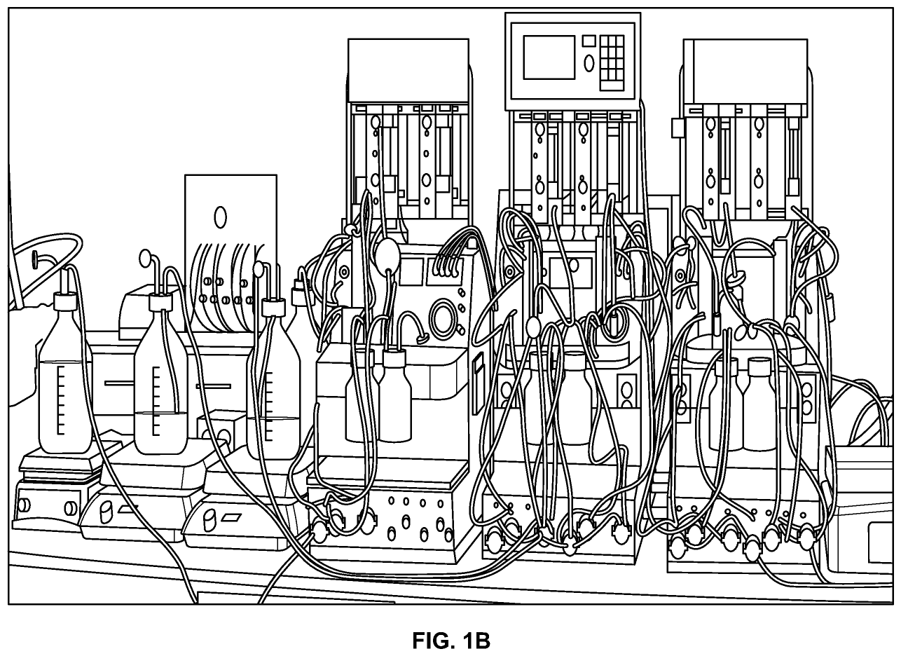

In some embodiments, the first and second compositions comprising a fecal-derived bacterial population are cultured in a chemostat vessel. In some embodiments, the chemostat vessel is the vessel disclosed in U.S. Patent Application Publication No. 20140342438. In some embodiments, the chemostat vessel is the vessel described in FIGS. 1A and 1B.

In some embodiments, the chemostat vessel was converted from a fermentation system to a chemostat by blocking off the condenser and bubbling nitrogen gas through the culture. In some embodiments, the pressure forces the waste out of a metal tube (formerly a sampling tube) at a set height and allows for the maintenance of given working volume of the chemostat culture.

In some embodiments, the chemostat vessel is kept anaerobic by bubbling filtered nitrogen gas through the chemostat vessel. In some embodiments, temperature and pressure are automatically controlled and maintained

In some embodiments, the culture pH of the chemostat culture is maintained using 5% (v/v) HCl (Sigma) and 5% (w/v) NaOH (Sigma).

In some embodiments, the culture medium of the chemostat vessel is continually replaced. In some embodiments, the replacement occurs over a period of time equal to the retention time of the distal gut. Consequently, in some embodiments, the culture medium is continuously fed into the chemostat vessel at a rate of 400 mL/day (16.7 mL/hour) to give a retention time of 24 hours, a value set to mimic the retention time of the distal gut. An alternate retention time can be 65 hours (approximately 148 mL/day, 6.2 mL/hour). In some embodiments, the retention time can be as short as 12 hours.

In some embodiments, the culture medium is a culture medium disclosed in U.S. Patent Application Publication No. 20140342438.

In some embodiments, the chemostat is seeded with the second composition comprising a fecal-derived bacterial population, and the second composition comprising a fecal-derived bacterial population is cultured for 24 hours, prior to addition of the first composition comprising a fecal-derived bacterial population.

In some embodiments, the first composition comprising a fecal-derived bacterial population is a live culture.

In some embodiments, the first culture of the first composition comprising a fecal-derived bacterial population is cultured with the second composition comprising a fecal-derived bacteria population in the chemostat for a time sufficient to expand the at least one bacterial strain, the presence of which cannot be determined without further expansion to an amount that can be detected. In some embodiments, the time sufficient to expand the at least one bacterial strain, the presence of which cannot be determined without further expansion to an amount that can be detected is greater than 14 days. In some embodiments, the time sufficient to expand the at least one bacterial strain, the presence of which cannot be determined without further expansion to an amount that can be detected is 14 days. In some embodiments, the time sufficient to expand the at least one bacterial strain, the presence of which cannot be determined without further expansion to an amount that can be detected is 13 days. In some embodiments, the time sufficient to expand the at least one bacterial strain, the presence of which cannot be determined without further expansion to an amount that can be detected is 12 days. In some embodiments, the time sufficient to expand the at least one bacterial strain, the presence of which cannot be determined without further expansion to an amount that can be detected is 11 days. In some embodiments, the time sufficient to expand the at least one bacterial strain, the presence of which cannot be determined without further expansion to an amount that can be detected is 10 days. In some embodiments, the time sufficient to expand the at least one bacterial strain, the presence of which cannot be determined without further expansion to an amount that can be detected is 9 days. In some embodiments, the time sufficient to expand the at least one bacterial strain, the presence of which cannot be determined without further expansion to an amount that can be detected is 8 days. In some embodiments, the time sufficient to expand the at least one bacterial strain, the presence of which cannot be determined without further expansion to an amount that can be detected is 7 days. In some embodiments, the time sufficient to expand the at least one bacterial strain, the presence of which cannot be determined without further expansion to an amount that can be detected is 6 days. In some embodiments, the time sufficient to expand the at least one bacterial strain, the presence of which cannot be determined without further expansion to an amount that can be detected is 5 days. In some embodiments, the time sufficient to expand the at least one bacterial strain, the presence of which cannot be determined without further expansion to an amount that can be detected is 4 days. In some embodiments, the time sufficient to expand the at least one bacterial strain, the presence of which cannot be determined without further expansion to an amount that can be detected is 3 days. In some embodiments, the time sufficient to expand the at least one bacterial strain, the presence of which cannot be determined without further expansion to an amount that can be detected is 2 days. In some embodiments, the time sufficient to expand the at least one bacterial strain, the presence of which cannot be determined without further expansion to an amount that can be detected is 1 day.

### Identification of the at Least One Bacterial Strain at a Level Below a Threshold for Detection According to Some Embodiments of the Present Invention

In some embodiments, the at least one bacterial strain at a level below a threshold for detection is identified using a method disclosed in U.S. Patent Application No. 20140342438. Alternatively, in some embodiments, the at least one bacterial strain at a level below a threshold for detection is identified using a method disclosed in U.S. Patent Application No. 20140363397.

In some embodiments, the at least one bacterial strain at a level below a threshold for detection is identified using the polymerase chain reaction-based methods and subsequent analysis of denaturing gradient gel electrophoresis (DGGE) disclosed in U.S. Patent Application No. 20140342438.

In some embodiments, the least one bacterial strain at a level below a threshold for detection is identified using next generation sequencing methods. In some embodiments, the increased sensitivity of next generation sequencing methods enables the least one bacterial strain at a level below a threshold for detection to be identified with a shorter time in culture, compared to other detection methods. In some embodiments, the increased sensitivity of next generation sequencing methods enables the least one bacterial strain at a level below a threshold for detection to be identified with a lesser amount of sample material, compared to other detection methods.

In some embodiments, the at least one bacterial strain at a level below a threshold for detection is identified using the phylogenetic analysis of the 16S rRNA gene. In some embodiments, the 16S rRNA gene is amplified via a polymerase chain reaction using nucleic acid primers having a nucleotide sequence selected from the group consisting of: TACGG[AG]AGGCAGCAG (V31k primer, position 343-357 of E. coli 16S rRNA gene), and AC[AG]ACACGAGCTGACGAC (V6r primer, position 1078-1061 of E. coli 16S rRNA gene).

In some embodiments, the at least one bacterial strain at a level below a threshold for detection is identified using the phylogenetic analysis of the 16S rRNA gene according to the methods described in International Patent Application Publication No. WO2012045150.

In some embodiments, the 16S rRNA gene is amplified via a polymerase chain reaction and the sequence of the amplified genes are subsequently sequenced. In some embodiments, the V3k1 V6r region of the 16S rRNA gene is sequenced.

In some embodiments, a first phylogenetic profile is obtained. In some embodiments, the first phylogenetic profile is obtained via the phylogenetic analysis of the 16S rRNA gene of the bacterial strains in the first composition comprising a fecal-derived bacterial population is performed, prior to culturing the first composition comprising a fecal-derived bacterial population with the second composition comprising a fecal-derived bacterial population in the chemostat for a time sufficient to expand the at least one bacterial strain at a level below a threshold for detection above the threshold level for detection.

In some embodiments, the first phylogenetic profile is known.

In some embodiments, a second phylogenetic profile is obtained. In some embodiments, the second phylogenetic profile is obtained via the phylogenetic analysis of the 16S rRNA gene of the bacterial strains in the second composition comprising a fecal-derived bacterial population is performed, prior to culturing the first composition comprising a fecal-derived bacterial population with the second composition comprising a fecal-derived bacterial population in the chemostat for a time sufficient to expand the at least one bacterial strain at a level below a threshold for detection above the threshold level for detection.

In some embodiments, the second phylogenetic profile is known.

In some embodiments, a third phylogenetic profile is obtained. In some embodiments, the third phylogenetic profile is obtained via the phylogenetic analysis of the 16S rRNA gene of the bacterial strains in the chemostat culture medium after culturing the first composition comprising a fecal-derived bacterial population with the second composition comprising a fecal-derived bacterial population in the chemostat for a time sufficient to expand the at least one bacterial strain at a level below a threshold for detection above the threshold level for detection.

In some embodiments, the third phylogenetic profile is obtained from a sample comprising chemostat culture medium removed from the chemostat after the first culture of the first composition comprising a fecal-derived bacterial population has been cultured with the second composition comprising a fecal-derived bacterial population in the chemostat for a time sufficient to expand the at least one bacterial strain, the presence of which cannot be determined without further expansion to an amount that can be detected. In some embodiments, the time sufficient to expand the at least one bacterial strain, the presence of which cannot be determined without further expansion to an amount that can be detected is greater than 14 days. In some embodiments, the time sufficient to expand the at least one bacterial strain, the presence of which cannot be determined without further expansion to an amount that can be detected is 14 days. In some embodiments, the time sufficient to expand the at least one bacterial strain, the presence of which cannot be determined without further expansion to an amount that can be detected is 13 days. In some embodiments, the time sufficient to expand the at least one bacterial strain, the presence of which cannot be determined without further expansion to an amount that can be detected is 12 days. In some embodiments, the time sufficient to expand the at least one bacterial strain, the presence of which cannot be determined without further expansion to an amount that can be detected is 11 days. In some embodiments, the time sufficient to expand the at least one bacterial strain, the presence of which cannot be determined without further expansion to an amount that can be detected is 10 days. In some embodiments, the time sufficient to expand the at least one bacterial strain, the presence of which cannot be determined without further expansion to an amount that can be detected is 9 days. In some embodiments, the time sufficient to expand the at least one bacterial strain, the presence of which cannot be determined without further expansion to an amount that can be detected is 8 days. In some embodiments, the time sufficient to expand the at least one bacterial strain, the presence of which cannot be determined without further expansion to an amount that can be detected is 7 days. In some embodiments, the time sufficient to expand the at least one bacterial strain, the presence of which cannot be determined without further expansion to an amount that can be detected is 6 days. In some embodiments, the time sufficient to expand the at least one bacterial strain, the presence of which cannot be determined without further expansion to an amount that can be detected is 5 days. In some embodiments, the time sufficient to expand the at least one bacterial strain, the presence of which cannot be determined without further expansion to an amount that can be detected is 4 days. In some embodiments, the time sufficient to expand the at least one bacterial strain, the presence of which cannot be determined without further expansion to an amount that can be detected is 3 days. In some embodiments, the time sufficient to expand the at least one bacterial strain, the presence of which cannot be determined without further expansion to an amount that can be detected is 2 days. In some embodiments, the time sufficient to expand the at least one bacterial strain, the presence of which cannot be determined without further expansion to an amount that can be detected is 1 day.

In some embodiments, the first phylogenetic profile is analyzed to determine the operational taxonomic units within the first composition comprising a fecal-derived bacterial population.

In some embodiments, the second phylogenetic profile is analyzed to determine the operational taxonomic units within the second composition comprising a fecal-derived bacterial population.

In some embodiments, the third phylogenetic profile is analyzed to determine the operational taxonomic units within the chemostat culture medium after culturing the first composition comprising a fecal-derived bacterial population with the second composition comprising a fecal-derived bacterial population in the chemostat for a time sufficient to expand the at least one bacterial strain at a level below a threshold for detection above the threshold level for detection

In some embodiments, relative abundance of the bacterial strains in the determined operational taxonomic units is calculated.

In some embodiments, the third phylogenetic profile is compared to either the first phylogenetic profile, or the second phylogenetic profile, or both the first phylogenetic profile and second phylogenetic profile. Any 16S rRNA sequences present in the third phylogenetic profile, that are not present in either the first phylogenetic profile, or the second phylogenetic profile, or both the first phylogenetic profile and second phylogenetic profile are selected and used to identify bacterial strains that have been expanded to a level above the threshold for detection.

In some embodiments, the identified bacterial strains that have been expanded to a level above the threshold for detection are subsequently isolated and purified. In some embodiments, a pure culture is generated of the identified bacterial strains that have been expanded to a level above the threshold for detection.

In some embodiments, the pure culture is obtained according to the methods disclosed in U.S. Patent Application Publication No. 20140342438.

In some embodiments, the pure culture is obtained by culturing a sample comprising chemostat culture medium removed from the chemostat after the first culture of the first composition comprising a fecal-derived bacterial population has been cultured with the second composition comprising a fecal-derived bacterial population in the chemostat for a time sufficient to expand the at least one bacterial strain, under conditions selective for the identified bacterial strains that have been expanded to a level above the threshold for detection. In some embodiments, the selective condition is a specific carbon source. In some embodiments, the selective condition is antibiotic resistance.

In some embodiments, the present invention provides a phylogenetic profile of a fecal-derived bacterial population. In some embodiments, the present invention provides a metabolic profile of a fecal-derived bacterial population. In some embodiments, the metabolic profile comprises the chemical constituents present in the medium in which the fecal-derived bacterial population has been cultured. In some embodiments, the present invention provides a genomic profile of the fecal-derived bacterial population.

In some embodiments, the phylogenetic profile is used to determine the identity of the bacterial strains within the first composition comprising a fecal-derived bacterial population. In some embodiments, the phylogenetic profile is used to determine if the bacterial strains within the first composition comprising a fecal-derived bacterial population changes with time.

In some embodiments, the phylogenetic profile is used to determine the identity of the bacterial strains within the second composition comprising a fecal-derived bacterial population. In some embodiments, the phylogenetic profile is used to determine if the bacterial strains within the second composition comprising a fecal-derived bacterial population changes with time.

In some embodiments, the metabolic profile of the culture medium is used to determine the identity of the bacterial strains within the first composition comprising a fecal-derived bacterial population. In some embodiments, the metabolic profile of the culture medium is used to determine if the bacterial strains within the first composition comprising a fecal-derived bacterial population changes with time.

In some embodiments, the metabolic profile of the culture medium is used to determine the identity of the bacterial strains within the second composition comprising a fecal-derived bacterial population. In some embodiments, the metabolic profile of the culture medium is used to determine if the bacterial strains within the second composition comprising a fecal-derived bacterial population changes with time.

In some embodiments, the metabolic profile is obtained by culturing a fecal-derived bacterial population in the chemostat vessel, in a defined culture medium (i.e., a culture medium with known constituents). In some embodiments, the constituents of the defined culture medium will change, and the extent, and the particular constituents that change depend on the particular fecal-derived bacterial population. In this manner, the identity, purity or contaminant present within the fecal-derived bacterial population may be determined by assaying the change in metabolites in the defined medium, or by examining the metabolite profile of the medium after culture in the conditioned medium.

In some embodiments, the fecal-derived bacterial population may be altered intentionally, to add, or remove a particular metabolite from the culture medium. The modification may be the addition, or removal of certain microbial strains from the fecal-derived bacterial population. For example, by way of illustration, microbial strains known to produce butyrate may be added to the fecal-derived bacterial population, if increased levels of butyrate in the culture medium is required. In another example, microbial strains that produce harmful metabolites may be removed from the fecal-derived bacterial population.

In another example, in some embodiments, the metabolite profile may be used as an assay to determine the effects of a particular diet, drug on a particular fecal-derived bacterial population.

In some embodiments, the metabolic profile comprises at least one metabolite disclosed in Yen et al., J. Proteome Res. 2015, 14, 1472-1482.

In some embodiments, the metabolic profile is determined via nuclear magnetic resonance, according to the methods disclosed in Yen et al., J. Proteome Res. 2015, 14, 1472-1482.

In some embodiments, a cell-free supernatant is obtained from a culture of the fecal-derived bacterial population, and spectra obtained using ¹H nuclear magnetic resonance (NMR) spectroscopy. In some embodiments, the spectra are representative of the metabolic activity of the fecal-derived bacterial population.

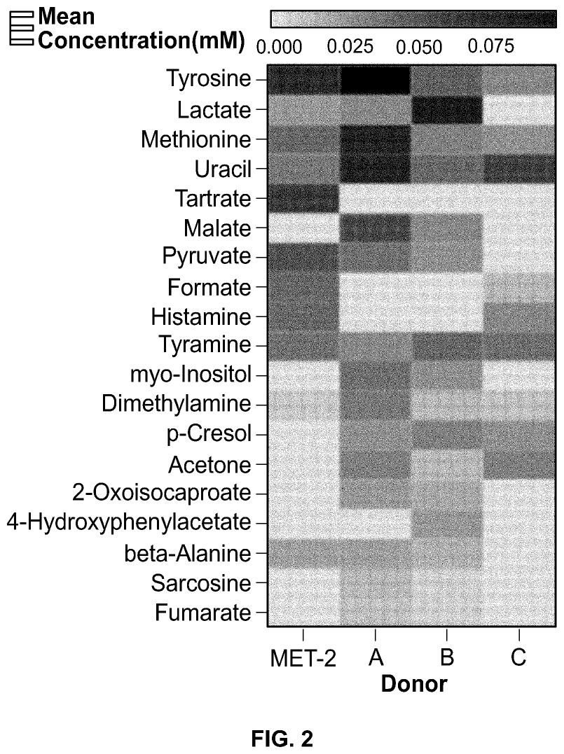

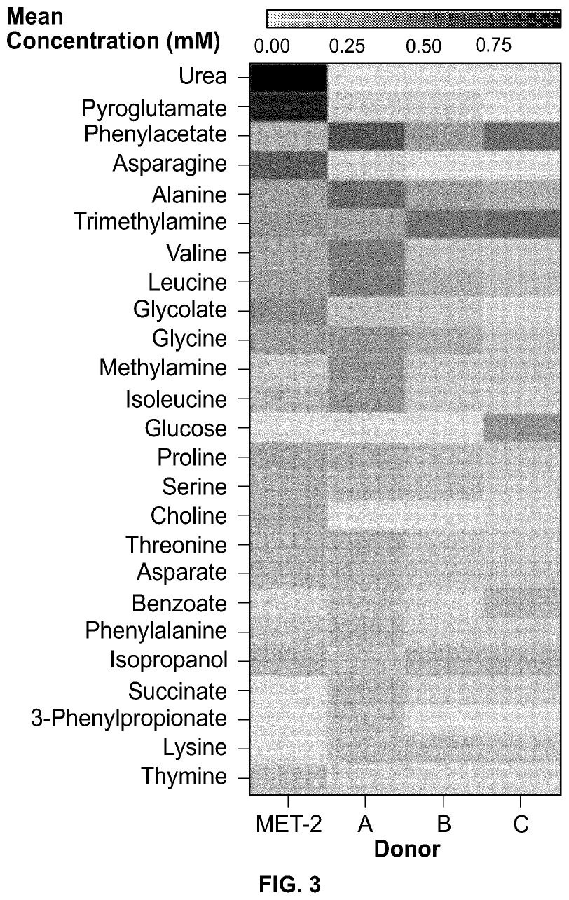

In some embodiments, the spectra are analyzed and specific metabolites are identified and quantified. In some embodiments, the identified metabolites are used to generate the metabolic profile of the fecal-derived bacterial population. Examples of metabolic profiles according to some embodiments of the present invention are shown in FIGS. 2 and 3.

In some embodiments, the metabolic profile is determined via GC-MS (gas chromatography-mass spectrometry), according to the methods disclosed in Garner et al., FASEB J 21: 1675-1688 (2007).

In some embodiments, a cell-free supernatant is obtained from a culture of the fecal-derived bacterial population, and spectra obtained using GC-MS. In some embodiments, the spectra are representative of the metabolic activity of the fecal-derived bacterial population.

In some embodiments, the spectra are analyzed and specific metabolites are identified and quantified. In some embodiments, the identified metabolites are used to generate the metabolic profile of the fecal-derived bacterial population.

In some embodiments, the genomic profile of the culture medium is used to determine the identity of the bacterial strains within the first composition comprising a fecal-derived bacterial population. In some embodiments, the genomic profile of the culture medium is used to determine if the bacterial strains within the first composition comprising a fecal-derived bacterial population changes with time.

In some embodiments, the genomic profile of the culture medium is used to determine the identity of the bacterial strains within the second composition comprising a fecal-derived bacterial population. In some embodiments, the genomic profile of the culture medium is used to determine if the bacterial strains within the second composition comprising a fecal-derived bacterial population changes with time.

While a number of embodiments of the present invention have been described, it is understood that these embodiments are illustrative only, and not restrictive, and that many modifications may become apparent to those of ordinary skill in the art. Further still, the various steps may be carried out in any desired order (and any desired steps may be added and/or any desired steps may be eliminated).

Reference is now made to the following examples, which together with the above descriptions illustrate some embodiments of the invention in a non limiting fashion.

### EXAMPLES

### Example 1: Determination of the Purity of a Fecal-Derived Bacterial Population

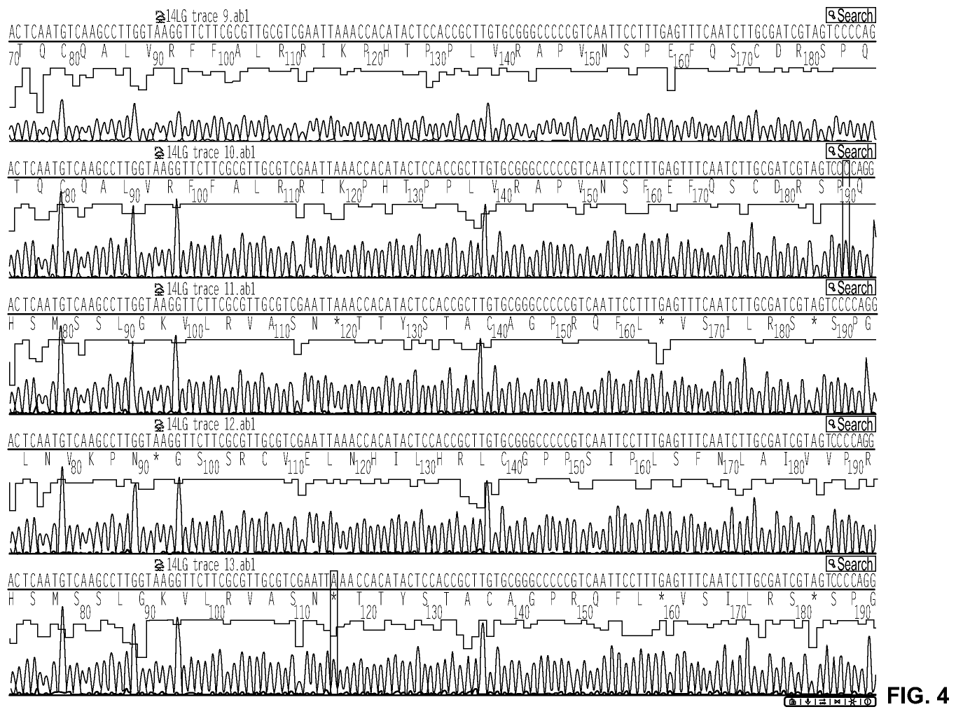

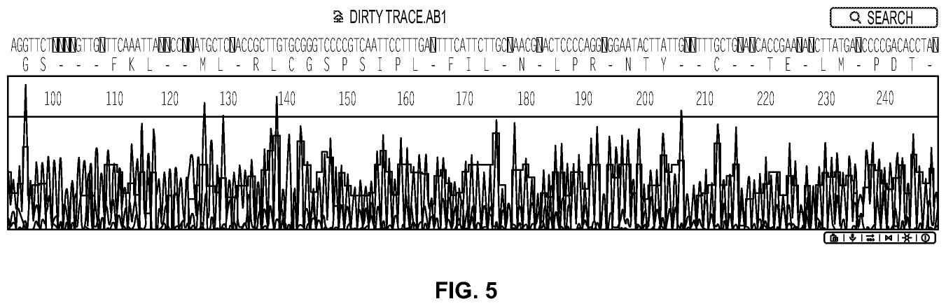

A fecal-derived bacterial population, hereinafter referred to as MET-1+ was obtained. Analysis based on the uniform colony morphology of the strain on Fastidious Anaerobe Agar (FAA) grown strictly anaerobically, and on obtaining a clean 16S full length Sanger sequence, initially indicated that a bacterial strain identified by the 16S rRNA sequence 16-6-S 14 LG, to be a pure strain Acidaminococcus intestini, i.e., a relative abundance of 100%. FIG. 4 shows sequence data obtained 16S rRNA profiling via the Sanger sequencing method. The traces show minimal noise, purportedly indicating a pure strain. By way of comparison, FIG. 5 shows sequence data obtained 16S rRNA profiling via the Sanger sequencing method from a contaminated strain. The traces show a large amount of noise, where the software is unable to resolve the individual peaks, thus being unable to define the nucleotide corresponding to the peak.

A live culture of MET-1+ was added to a chemostat seeded with a defined microbial community derived from the fecal sample of an Ulcerative Colitis (UC) patient (referred to as UC-3). The objective of these experiments was to see if the MET-1+ formulation would alter the dysbiotic microbial community of a UC patient. Samples of the chemostat vessel contents were taken before treatment, during MET-1+ treatment, and days 7 and 14 post-treatment, respectively. These samples were subjected to 16S rRNA profiling via the Illumina Miseq platform, and were subsequently analyzed using the Mothur program.

Unexpectedly, there was a significant expansion of microbes belonging to genus that were not present in either the MET-1+ formulation or the UC-3 microbial community, which occurred in the post-treatment samples. These genera were identified as Akkermansia, Flavonifractor and Sutterella. This expansion occurred in two separate experimental runs, despite the MET-1+ formulation isolates being carefully scrutinized for purity prior to addition.

Table 1 shows the relative abundance profiles of classified OTUs generated utilizing the Mothur pipeline. Shown below are three replicates of the before treatment sample (Before R#), three replicates of the day 14 post-treatment sample (After R#), and the total percentage of the OTU abundance across all samples. A total percentage of less than 0.01% is deemed insignificant (the cut-off used to remove sequencing error). The three OTUs of interest are bolded. Please note that these OTUs are only present in the post-treatment samples, and they are also above the 0.01% abundance cut-off

| | Before | Before | Before | After | After | After | % | |
|---|---|---|---|---|---|---|---|---|
| Group | R1 | R2 | R3 | R1 | R2 | R3 | Total | Taxonomy |
| Otu01 | 37 | 25 | 31 | 69985 | 34458 | 45443 | 10.13591 | Bacteria(100); |
| | | | | | | | | “Bacteroidetes”(100); |
| | | | | | | | | “Bacteroidia”(100); |
| | | | | | | | | “Bacteroidales”(100); |
| | | | | | | | | Bacteroidaceae(100); |
| | | | | | | | | Bacteroides(100); |
| Otu02 | 19911 | 22713 | 15168 | 2487 | 1460 | 1973 | 7.180915 | Bacteria(100); |
| | | | | | | | | “Proteobacteria”(100); |
| | | | | | | | | Gammaproteobacteria(100); |
| | | | | | | | | “Enterobacteriales”(100); |
| | | | | | | | | Enterobacteriaceae(100); |
| | | | | | | | | unclassified(100); |
| Otu03 | 12807 | 16200 | 9733 | 10351 | 5573 | 7535 | 4.271535 | Bacteria(100); |
| | | | | | | | | Firmicutes(100); |
| | | | | | | | | Clostridia(100); |
| | | | | | | | | Clostridiales(100); |
| | | | | | | | | Lachnospiraceae(100); |
| | | | | | | | | unclassified(100); |
| Otu04 | 168 | 174 | 85 | 1 | 1 | 1 | 0.047432 | Bacteria(100); |
| | | | | | | | | Firmicutes(100); |
| | | | | | | | | Clostridia(100); |
| | | | | | | | | Clostridiales(100); |
| | | | | | | | | Clostridiaceae1(100); |
| | | | | | | | | unclassified(100); |
| Otu05 | 13859 | 17092 | 10305 | 296 | 181 | 235 | 5.896149 | Bacteria(100); |
| | | | | | | | | Firmicutes(100); |
| | | | | | | | | Negativicutes(100); |
| | | | | | | | | Selenomonadales(100); |
| | | | | | | | | Veillonellaceae(100); |
| | | | | | | | | Veillonella(100); |
| Otu06 | 5 | 5 | 7 | 25274 | 12663 | 16741 | 3.690819 | Bacteria(100); |
| | | | | | | | | “Bacteroidetes”(100); |
| | | | | | | | | “Bacteroidia”(100); |
| | | | | | | | | “Bacteroidales”(100); |
| | | | | | | | | “Porphyromonadaceae”(100); |
| | | | | | | | | Parabacteroides(100); |
| Otu07 | 5697 | 7429 | 4319 | 8703 | 4619 | 4587 | 4.035524 | Bacteria(100); |
| | | | | | | | | Firmicutes(100); |
| | | | | | | | | Clostridia(100); |
| | | | | | | | | Clostridiales(100); |
| | | | | | | | | Lachnospiraceae(100); |
| | | | | | | | | ClostridiumXlVa(100); |
| Otu08 | 2 | 5 | 5 | 21695 | 12531 | 15237 | 3.340582 | Bacteria(100); |
| | | | | | | | | “Verrucomicrobia”(100); |
| | | | | | | | | “Verrucomicrobiae(100); |
| | | | | | | | | Verrucomicrobiales(100); |
| | | | | | | | | Verrucomicrobiaceae(100); |
| | | | | | | | | Akkermansia(100); |
| Otu09 | 5 | 6 | 7 | 20481 | 10619 | 12365 | 2.936301 | Bacteria(100); |
| | | | | | | | | Firmicutes(100); |
| | | | | | | | | Negativicutes(100); |
| | | | | | | | | Selenomonadales(100); |
| | | | | | | | | Acidaminococcaceae(100); |
| | | | | | | | | Acidaminococcus(100); |
| Otu10 | 3431 | 4495 | 2448 | 1129 | 615 | 668 | 2.792859 | Bacteria(100); |
| | | | | | | | | Firmicutes(100); |
| | | | | | | | | Clostridia(100); |
| | | | | | | | | Clostridiales(100); |
| | | | | | | | | Lachnospiraceae(100); |
| | | | | | | | | Dorea(100); |
| Otu11 | 6 | 2 | 10 | 3897 | 2115 | 2352 | 0.566344 | Bacteria(100); |
| | | | | | | | | Firmicutes(100); |
| | | | | | | | | Clostridia(100); |
| | | | | | | | | Clostridiales(100); |
| | | | | | | | | Lachnospiraceae(100); |
| | | | | | | | | Roseburia(100); |
| Otu12 | 22379 | 27578 | 16962 | 147 | 98 | 66 | 8.024022 | Bacteria(100); |
| | | | | | | | | Firmicutes(100); |
| | | | | | | | | Clostridia(100); |
| | | | | | | | | Clostridiales(100); |
| | | | | | | | | Clostridiaceae_l(100); |
| | | | | | | | | Clostridium_sensu_stricto(100); |
| Otu13 | 4 | 4 | 1 | 4 | 2 | 0 | 2.66163 | Bacteria(100); |
| | | | | | | | | Firmicutes(100); |
| | | | | | | | | Bacilli(100); |
| | | | | | | | | Lactobacillales(100); |
| | | | | | | | | Streptococcaceae(100); |
| | | | | | | | | Streptococcus(100); |
| Otu14 | 20 | 30 | 10 | 9 | 5 | 5 | 1.835458 | Bacteria(100); |
| | | | | | | | | “Proteobacteria”(100); |
| | | | | | | | | Gammaproteobacteria(100); |
| | | | | | | | | “Enterobacteriales”(100); |
| | | | | | | | | Enterobacteriaceae(100); |
| | | | | | | | | Escherichia_Shigella(100); |
| Otu15 | 1786 | 2139 | 1314 | 9 | 2 | 10 | 1.005103 | Bacteria(100); |
| | | | | | | | | Firmicutes(100); |
| | | | | | | | | Clostridia(100); |
| | | | | | | | | Clostridiales(100); |
| | | | | | | | | unclassified(100); |
| | | | | | | | | unclassified(100); |
| Otu16 | 755 | 786 | 549 | 186 | 99 | 158 | 1.054626 | Bacteria(100); |
| | | | | | | | | “Proteobacteria”(100); |
| | | | | | | | | Gammaproteobacteria(100); |
| | | | | | | | | “Enterobacteriales”(100); |
| | | | | | | | | Enterobacteriaceae(100); |
| | | | | | | | | Proteus(100); |
| Otu17 | 2 | 4 | 1 | 4227 | 1994 | 3050 | 0.62619 | Bacteria(100); |
| | | | | | | | | Firmicutes(100); |
| | | | | | | | | Negativicutes(100); |
| | | | | | | | | Selenomonadales(100); |
| | | | | | | | | Veillonellaceae(100); |
| | | | | | | | | Dialister(100); |
| Otu18 | 0 | 1 | 2 | 3500 | 2405 | 2844 | 0.591038 | Bacteria(100); |
| | | | | | | | | “Actinobacteria”(100); |
| | | | | | | | | Actinobacteria(100); |
| | | | | | | | | Coriobacteriales(100); |
| | | | | | | | | Coriobacteriaceae(100); |
| | | | | | | | | Collinsella(100); |
| Otu19 | 0 | 0 | 0 | 4142 | 1903 | 2862 | 0.600957 | Bacteria(100); |
| | | | | | | | | Firmicutes(100); |
| | | | | | | | | Clostridia(100); |
| | | | | | | | | Clostridiales(100); |
| | | | | | | | | Ruminococcaceae(100); |
| | | | | | | | | Faecalibacterium(100); |
| Otu20 | 7 | 5 | 6 | 0 | 2 | 2 | 0.522624 | Bacteria(100); |
| | | | | | | | | Firmicutes(100); |
| | | | | | | | | Bacilli(100); |
| | | | | | | | | Lactobacillales(100); |
| | | | | | | | | Lactobacillaceae(100); |
| | | | | | | | | Lactobacillus(100); |
| Otu21 | 8 | 2 | 3 | 139 | 53 | 5 | 0.049051 | Bacteria(100); |
| | | | | | | | | Firmicutes(100); |
| | | | | | | | | Clostridia(100); |
| | | | | | | | | Clostridiales(100); |
| | | | | | | | | Lachnospiraceae(100); |
| | | | | | | | | Lachnospiracea_incertae_sedis(100); |
| Otu22 | 0 | 0 | 1 | 1180 | 629 | 817 | 0.177244 | Bacteria(100); |
| | | | | | | | | Firmicutes(100); |
| | | | | | | | | Clostridia(100); |
| | | | | | | | | Clostridiales(100); |
| | | | | | | | | Lachnospiraceae(100); |
| | | | | | | | | Blautia(100); |
| Otu23 | 0 | 0 | 0 | 680 | 345 | 552 | 0.1064 | Bacteria(100); |
| | | | | | | | | Firmicutes(100); |
| | | | | | | | | Clostridia(100); |
| | | | | | | | | Clostridiales(100); |
| | | | | | | | | Romiuococcaceae(100); |
| | | | | | | | | Flavonifractor(100); |
| Otu24 | 1 | 0 | 0 | 1026 | 449 | 655 | 0.144049 | Bacteria(100); |
| | | | | | | | | Firmicutes(100); |
| | | | | | | | | Clostridia(100); |
| | | | | | | | | Clostridiales(100); |
| | | | | | | | | Eubacteriaceae(100); |
| | | | | | | | | unclassified(100); |
| Otu25 | 0 | 0 | 0 | 1277 | 667 | 915 | 0.192965 | Bacteria(100); |
| | | | | | | | | “Proteobacteria”(100); |
| | | | | | | | | Betaproteobacteria(100); |
| | | | | | | | | Burkholderiales(100); |
| | | | | | | | | Sutterellaceae(100); |
| | | | | | | | | Sutterella(100); |
| Otu26 | 131 | 163 | 90 | 32 | 14 | 11 | 0.773613 | Bacteria(100); |
| | | | | | | | | Firmicutes(100); |
| | | | | | | | | Bacilli(100); |
| | | | | | | | | Lactobacillales(100); |
| | | | | | | | | Enterococcaceae(100); |
| | | | | | | | | Enterococcus(100); |
| Otu27 | 0 | 0 | 0 | 0 | 0 | 0 | 0.001754 | Bacteria(100); |
| | | | | | | | | Firmicutes(100); |
| | | | | | | | | Bacilli(100); |
| | | | | | | | | Lactobacillales(100); |
| | | | | | | | | Enterococcaceae(100); |
| | | | | | | | | unclassified(100); |
| Otu28 | 0 | 0 | 0 | 318 | 231 | 270 | 0.055326 | Bacteria(100); |
| | | | | | | | | “Actinobacteria”(100); |
| | | | | | | | | Actinobacteria(100); |
| | | | | | | | | Bifidobacteriales(100); |
| | | | | | | | | Bifidobacteriaceae(100); |
| | | | | | | | | Bifidobacterium(100); |
| Otu29 | 23 | 43 | 23 | 251 | 158 | 242 | 0.100058 | Bacteria(100); |
| | | | | | | | | Firmicutes(100); |
| | | | | | | | | Erysipelotrichia(100); |
| | | | | | | | | Erysipelotrichales(100); |
| | | | | | | | | Erysipelotrichaceae(100); |
| | | | | | | | | Clostridium_XVIII(100); |
| Otu30 | 8 | 9 | 3 | 4 | 2 | 0 | 0.003643 | Bacteria(100); |
| | | | | | | | | Firmicutes(100); |
| | | | | | | | | Bacilli(100); |
| | | | | | | | | Bacillales(100); |
| | | | | | | | | Bacillaceae1(100); |
| | | | | | | | | Geobacillus(100); |
| Otu31 | 2 | 0 | 0 | 0 | 0 | 0 | 0.00108 | Bacteria(100); |
| | | | | | | | | Firmicutes(100); |
| | | | | | | | | Bacilli(100); |
| | | | | | | | | Lactobacillales(100); |
| | | | | | | | | unclassified(100); |
| | | | | | | | | unclassified(100); |
| Otu32 | 0 | 0 | 0 | 185 | 93 | 73 | 0.023749 | Bacteria(100); |
| | | | | | | | | Firmicutes(100); |
| | | | | | | | | Clostridia(100); |
| | | | | | | | | Clostridiales(100); |
| | | | | | | | | Lachnospiraceae(100); |
| | | | | | | | | Coprococcus(100); |
| Otu33 | 0 | 3 | 2 | 10 | 11 | 11 | 0.0056 | Bacteria(100); |
| | | | | | | | | Firmicutes(100); |
| | | | | | | | | Negativicutes(100); |
| | | | | | | | | Selenomonadales(100); |
| | | | | | | | | Acidaminococcaceae(100); |
| | | | | | | | | Phascolarctobacterium(100); |
| Otu34 | 0 | 2 | 0 | 0 | 1 | 0 | 0.000472 | Bacteria(100); |
| | | | | | | | | “Deinococcus-Thermus”(100); |
| | | | | | | | | Deinococci(100); |
| | | | | | | | | Thermales(100); |
| | | | | | | | | Thermaceae(100); |
| | | | | | | | | Thermus(100); |
| Otu35 | 0 | 0 | 0 | 0 | 0 | 0 | 0.00054 | Bacteria(100); |
| | | | | | | | | Firmicutes(100); |
| | | | | | | | | Bacilli(100); |
| | | | | | | | | Lactobacillales(100); |
| | | | | | | | | Lactobacillaceae(100); |
| | | | | | | | | unclassified(100); |
| Otu36 | 0 | 1 | 2 | 0 | 0 | 2 | 0.00054 | Bacteria(100); |
| | | | | | | | | Firmicutes(100); |
| | | | | | | | | Bacilli(100); |
| | | | | | | | | Bacillales(100); |
| | | | | | | | | Bacillaceae_1(100); |
| | | | | | | | | Anoxybacillus(100); |
| Otu37 | 0 | 0 | 0 | 0 | 0 | 0 | 0.000202 | Bacteria(100); |
| | | | | | | | | “Proteobacteria”(100); |
| | | | | | | | | Gammaproteobacteria(100); |
| | | | | | | | | Pseudomonadales(100); |
| | | | | | | | | Pseudomonadaceae(100); |
| | | | | | | | | unclassified(100); |
| Otu38 | 2 | 2 | 0 | 0 | 0 | 0 | 0.00027 | Bacteria(100); |
| | | | | | | | | Firmicutes(100); |
| | | | | | | | | Bacilli(100); |
| | | | | | | | | Lactobacillales(100); |
| | | | | | | | | Streptococcaceae(100); |
| | | | | | | | | Lactococcus(100); |
| Otu39 | 0 | 0 | 1 | 0 | 0 | 1 | 0.00027 | Bacteria(100); |
| | | | | | | | | Firmicutes(100); |
| | | | | | | | | Clostridia(100); |
| | | | | | | | | Clostridiales(100); |
| | | | | | | | | Ruminococcaceae(100); |
| | | | | | | | | unclassified(100); |
| Otu40 | 0 | 0 | 0 | 0 | 0 | 0 | 6.75E−05 | Bacteria(100); |
| | | | | | | | | “Proteobacteria”(100); |
| | | | | | | | | Gammaproteobacteria(100); |
| | | | | | | | | Pseudomonadales(100); |
| | | | | | | | | Moraxellaceae(100); |
| | | | | | | | | Acinetobacter(100); |
| Otu41 | 0 | 0 | 1 | 2 | 1 | 0 | 0.00027 | Bacteria(100); |
| | | | | | | | | “Proteobacteria”(100); |
| | | | | | | | | Gammaproteobacteria(100); |
| | | | | | | | | Pseudomonadales(100); |
| | | | | | | | | Pseudomonadaceae(100); |
| | | | | | | | | Pseudomonas(100); |
| Otu42 | 1 | 0 | 1 | 0 | 0 | 0 | 0.000135 | Bacteria(100); |
| | | | | | | | | “Proteobacteria”(100); |
| | | | | | | | | Betaproteobacteria(100); |
| | | | | | | | | Burkholderiales(100); |
| | | | | | | | | Oxalobacteraceae(100); |
| | | | | | | | | Massilia(100); |
| Otu43 | 6 | 0 | 0 | 2 | 0 | 0 | 0.00054 | Bacteria(100); |
| | | | | | | | | Firmicutes(100); |
| | | | | | | | | Bacilli(100); |
| | | | | | | | | Bacillales(100); |
| | | | | | | | | Bacillaceae_1(100); |
| | | | | | | | | unclassified(100); |
| Otu44 | 0 | 0 | 0 | 0 | 0 | 0 | 0 | Bacteria(100); |
| | | | | | | | | “Proteobacteria”(100); |
| | | | | | | | | Betaproteobacteria(100); |
| | | | | | | | | Burkholderiales(100); |
| | | | | | | | | Comamonadaceae(100); |
| | | | | | | | | Acidovorax(100); |
| Otu45 | 0 | 0 | 0 | 1 | 0 | 0 | 6.75E−05 | Bacteria(100); |
| | | | | | | | | “Bacteroidetes”(100); |
| | | | | | | | | “Bacteroidia”(100); |
| | | | | | | | | “Bacteroidales”(100); |
| | | | | | | | | “Porphyromonadaceae”(100); |
| | | | | | | | | unclassified(100); |
| Otu46 | 0 | 0 | 0 | 0 | 1 | 0 | 6.75E−05 | Bacteria(100); |
| | | | | | | | | Firmicutes(100); |
| | | | | | | | | Negativicutes(100); |
| | | | | | | | | Selenomonadales(100); |
| | | | | | | | | Veillonellaceae(100); |
| | | | | | | | | unclassified(100); |
| Otu47 | 0 | 0 | 0 | 0 | 0 | 0 | 0 | Bacteria(100); |
| | | | | | | | | Firmicutes(100); |
| | | | | | | | | Negativicutes(100); |
| | | | | | | | | Selenomonadales(100); |
| | | | | | | | | Veillonellaceae(100); |
| | | | | | | | | unclassified(100); |
| Otu48 | 0 | 0 | 0 | 0 | 0 | 0 | 0.000135 | Bacteria(100); |
| | | | | | | | | Firmicutes(100); |
| | | | | | | | | Bacilli(100); |
| | | | | | | | | unclassified(100); |
| | | | | | | | | unclassified(100); |
| | | | | | | | | unclassified(100); |
| Otu49 | 0 | 0 | 0 | 0 | 0 | 0 | 0 | Bacteria(100); |
| | | | | | | | | unclassified(100); |
| | | | | | | | | unclassified(100); |
| | | | | | | | | unclassified(100); |
| | | | | | | | | unclassified(100); |
| | | | | | | | | unclassified(100); |
| Otu50 | 0 | 0 | 0 | 0 | 0 | 0 | 6.75E−05 | Bacteria(100); |
| | | | | | | | | “Proteobacteria”(100); |
| | | | | | | | | Alphaproteobacteria(100); |
| | | | | | | | | Caulobacterales(100); |
| | | | | | | | | Caulobacteraceae(100); |
| | | | | | | | | Brevundimonas(100); |
| Otu51 | 0 | 0 | 0 | 0 | 0 | 2 | 0.000135 | Bacteria(100); |
| | | | | | | | | “Proteobacteria”(100); |
| | | | | | | | | Gammaproteobacteria(100); |
| | | | | | | | | Legionellales(100); |
| | | | | | | | | Coxiellaceae(100); |
| | | | | | | | | Aquicella(100); |
| Otu52 | 0 | 0 | 0 | 0 | 0 | 0 | 0.000135 | Bacteria(100); |
| | | | | | | | | “Bacteroidetes”(100); |
| | | | | | | | | “Sphingobacteria”(100); |
| | | | | | | | | “Sphingobacteriales”(100); |
| | | | | | | | | Chitinophagaceae(100); |
| | | | | | | | | unclassified(100); |
| Otu53 | 0 | 0 | 0 | 0 | 0 | 0 | 0 | Bacteria(100); |
| | | | | | | | | Firmicutes(100); |
| | | | | | | | | Bacilli(100); |
| | | | | | | | | Lactobacillales(100); |
| | | | | | | | | Leuconostocaceae(100); |
| | | | | | | | | Leuconostoc(100); |
| Otu54 | 0 | 0 | 0 | 0 | 0 | 0 | 6.75E−05 | Bacteria(100); |
| | | | | | | | | “Proteobacteria”(100); |
| | | | | | | | | Betaproteobacteria(100); |
| | | | | | | | | Burkholderiales(100); |
| | | | | | | | | Oxalobacteraceae(100); |
| | | | | | | | | unclassified(100); |
| Otu55 | 0 | 0 | 0 | 0 | 1 | 0 | 6.75E−05 | Bacteria(100); |
| | | | | | | | | “Actinobacteria”(100); |
| | | | | | | | | Actinobacteria(100); |
| | | | | | | | | Coriobacteriales(100); |
| | | | | | | | | Coriobacteriaceae(100); |
| | | | | | | | | Asaccharobacter(100); |
| Otu56 | 0 | 0 | 0 | 0 | 0 | 0 | 6.75E−05 | Bacteria(100); |
| | | | | | | | | “Proteobacteria”(100); |
| | | | | | | | | Gammaproteobacteria(100); |
| | | | | | | | | “Enterobacteriales”(100); |
| | | | | | | | | Enterobacteriaceae(100); |
| | | | | | | | | Enterobacter(100); |
| Otu57 | 0 | 0 | 1 | 0 | 0 | 0 | 6.75E−05 | Bacteria(100); |
| | | | | | | | | “Bacteroidetes”(100); |
| | | | | | | | | “Sphingobacteria”(100); |
| | | | | | | | | “Sphingobacteriales”(100); |
| | | | | | | | | Sphingobacteriaceae(100); |
| | | | | | | | | Mucilaginibacter(100); |

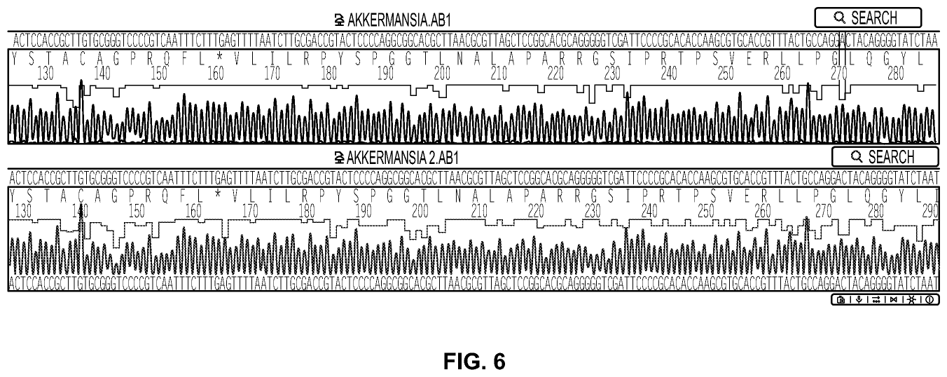

The largest unexpected expansion was of Akkermansia, which at the time was not known to be preset within the defined microbial community of the donor from which the MET-1+ formulation was derived. FIG. 6 shows sequence data obtained 16S rRNA profiling via the Sanger sequencing method of the expanded Akkermansia strain. The trace indicates a pure culture.

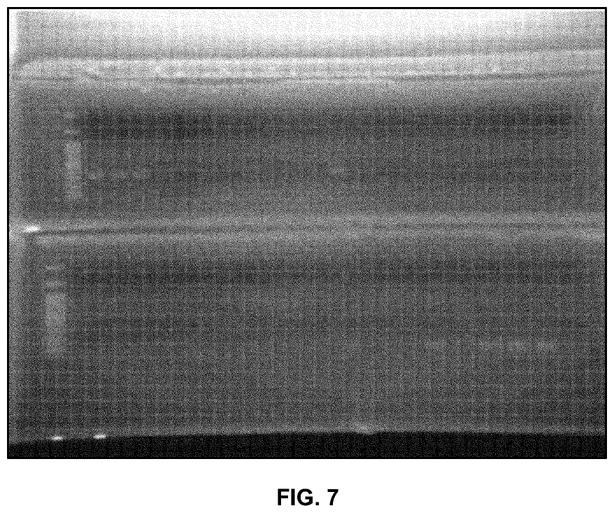

All stocks of the MET-1+ formulation were screened for the presence of Akkermansia via PCR using a set of Akkermansia specific primers. The resulting agarose gel stained with ethidium bromide is depicted below, with the positive controls comprising the first three samples after the 100 by DNA ladder, in FIG. 7.

Referring to FIG. 7, the visible band corresponded to the stock 16-6-S 14 LG. Once identified, attempts to isolate the Akkermansia were undertaken. As Akkermansia muciniphila is known to use mucin as a carbon source, three types of medium were used in attempt to isolate the species, the basal medium both with and without vitamin supplementation described by Derrien et al. (2004), and FAA supplemented with 0.4% mucin (referred to as Fmu).

These initial attempts were unsuccessful, thus in addition to enrichment, relative antibiotic resistance was exploited. Based on the antibiotic resistance profile of an Akkermansia muciniphila strain isolated from another donor fecal sample in the Allen-Vercoe laboratory, 5 1.tg Moxifloxacin BBL Sensi Discs were placed on Fmu medium immediately after sub-culturing. Meticulous re-streaking technique not only yielded pure culture of the isolate suspected to be Akkermansia muciniphila, but also yielded three other isolates of unique colony morphology. These isolates were subjected to 16S rRNA Sanger sequencing of the 16S V3k1 V6r region. Through this method, Akkermansia muciniphila was isolated, and the three other isolates were determined to be Eubacterium limosum (already present in the MET-1+ formulation), Flavonifractor plautii and Sutterella stercoricanis. The Flavonifractor and Sutterella were already known to be present in the defined microbial community of the donor from which the MET-1+ formulation was derived.

Table 2 shows isolates from 16-6-S 14 LG stock of the MET-1+ formulation, identified by 16S rRNA Sanger sequencing of the V3k1 V6r region, following culture according to methods of some embodiments of the present invention.

| Isolate # | Taxonomy | % ID | .abl file |
|---|---|---|---|
| 1 | Flavonifractor plautii | 94% | 1_E07.ab 1 |
| 2 | Sutterella stercoricanis | 88%* | AX1_D09.abl |
| 3 | Akkermansia muciniphila | 99% | AX2_D07.abl |
| 4 | Acidaminococcus intestini | 99% | OR-W_C09.ab1 |
| 5 | Eubacterium limosum | 98% | OR-YF_B09.ab1 |

In conclusion, 16-6-S 14 LG of the MET-1+ formulation, originally thought to be a pure culture of Acidaminococcus intestini, was found to be contaminated with four different microbial strains, which were successfully isolated: Eubacterium limosum, Flavonifractor plautii, Sutterella stercoricanis and Akkermansia muciniphila. Eubacterium limosum is part of the MET-1+ formulation. Flavonifractor plautii and Sutterella stercoricanis were known to be present in the defined community of the donor from which the MET-1+ formulation was derived. Akkermansia muciniphila was not known to be present in either community. The isolation of the latter three strains corresponds to the unexpected results obtained in the set of chemostat system experiments.

### Example 2: Determination of the Metabolite Profile of Fecal-Derived Bacterial Populations

Two fecal-derived bacterial populations, hereinafter referred to as MET-1 and MET-2 were obtained and cultured separately according to the methods described in U.S. Patent Application Publication No. 20140342438. The MET-1 population is described in U.S. Patent Application No. 20140363397. The MET-2 population is described in Yen et al., J. Proteome Res. 2015, 14, 1472-1482. The metabolite profile was determined via NMR, prior to culture. The two fecal-derived bacterial populations were cultured for 10 days, and the metabolite profile was determined via NMR, according to the methods disclosed in Yen et al., J. Proteome Res. 2015, 14, 1472-1482. The results are shown in Table 3. From Table 3, both fecal derived bacterial populations added metabolites to the culture medium. Additionally, some metabolites were depleted from the medium. These data suggest that the “metabolite signature” obtained following culture under defined conditions, such as, for example, in the chemostat vessel according to the methods described in U.S. Patent Application Publication No. 20140342438, may be used to identify the particular bacterial population. Alternatively, the “metabolite signature” obtained following culture under defined conditions, such as, for example, in the chemostat vessel according to the methods described in U.S. Patent Application Publication No. 20140342438, may be used to determine if the integrity of the bacterial population.

| Media compounds | | MET1 | MET2 |
|---|---|---|---|
| (no. metabolites | | (no. metabolites | (no. metabolites |
| profiled = 29) | change | profiled = 56) | profiled = 41) |
| Acetate | added | 5,6-Dihydrouracil | Asparagine |
| Adenosine | | Arabinose | Benzoate |
| Alanine | | Asparagine | Choline |
| Aspartate | | Benzoate | Fructose |
| Betaine | | Choline | Fumarate |
| Butyrate | | Fructose | Galactose |
| Cystine | | Fumarate | Glucose |
| Ethanol | | Galactose | Glycerol |
| Formate | | Histidine | Isobutyrate |
| Glutamate | | Isobutyrate | Isovalerate |
| Glycine | | Isovalerate | Methylamine |
| Glycolate | | Phenylacetate | Phenylacetate |
| Hydroxyacetone | | Propionate | Propionate |
| Isoleucine | | Pyruvate | Thymine |
| Lactate | | Thymine | Trimethylamine |
| Leucine | | Trimethylamine | Uracil |
| Lysine | | Uracil | Valerate |
| Methanol | | Valerate | Xylose |
| Methionine | | beta-Alanine | |
| Phenylalanine | | Acetone | |
| Proline | | Ethanolamine | |
| Pyroglutamate | | Glycerol | |
| Serine | | Histamine | |
| Succinate | | Isopropanol | |
| Threonine | | Methylamine | |
| Trehalose | | N,N-Dimethylglycine | |
| Tryptophan | | Sarcosine | |
| Tyrosine | | Tyramine | |
| Valine | | Urea | |
| | | Xanthine myo-Inositol | |
| | depleted | Adenosine | Adenosine |
| | | Cystine | Cystine |
| | | Trehalose | Formate |
| | | Tryptophan | Lactate |
| | | Glutamate | Trehalose |
| | | Pyroglutamate | Tryptophan |
| | | | Lysine |
| | | | Succinate |

### Example 3: Determination of the Metabolite Profile of Fecal-Derived Bacterial Populations

Two fecal-derived bacterial populations, hereinafter referred to as MET-1 and MET-2 were obtained and cultured separately according to the methods described in U.S. Patent Application Publication No. 20140342438. The MET-1 population is described in U.S. Patent Application No. 20140363397. The MET-2 population is described in Yen et al., J. Proteome Res. 2015, 14, 1472-1482. The metabolite profile was determined via NMR, prior to culture. The two fecal-derived bacterial populations were cultured for the times indicated in Table 4, and the metabolite profile was determined via NMR, according to the methods disclosed in Yen et al., J. Proteome Res. 2015, 14, 1472-1482. The concentration of the metabolites in mM are shown in Table 4.

Referring to Table 4, for a given fecal-derived bacterial population, and for a given period of culture, there is little variation in the observed concentrations of the metabolites detected. However, the metabolites detected and their concentration (i.e., the metabolite profile) differed between the two fecal-derived bacterial populations tested. For example, Alanine is at a 1.1-1.15 range for MET-1 but only a 0.20-0.21 range for MET-2. Another example is that Pyroglutamate is not detected in MET-1 but is detected at 0.8-1.3 range in MET-2. These data suggest that the metabolic profile of a fecal-derived bacterial population may be useful to identify, quantify, or determine the purity of the fecal-derived bacterial population.

| | | | | | | | | | | | |
|---|---|---|---|---|---|---|---|---|---|---|---|
| id | expt | donor | day | run | 5,6Di [symbol] | Acetate | Alanine | Arabinose | Asparagine | Aspartate | Benzoate |

Two runs of ecosystem MET-1

| | | | | | | | | | | | |
|---|---|---|---|---|---|---|---|---|---|---|---|
| 1 AF | RP1 | MET1 | Week 2 | 6 | 0.0518 | 40.1896 | 1.1572 | NA | 0.7221 | 0.0784 | 0.0126 |
| 2 B | [symbol] | MET1 | Week 2 | 1 | 0.05822 | 40.92011 | 1.099444 | 0.149111 | 014911 | 0.259444 | 0. [symbol] |

Three runs of ecosystem MET2

| | | | | | | | | | | | |
|---|---|---|---|---|---|---|---|---|---|---|---|
| 3 C | biomass | MET2 | Week 3 | 32 | NA | 82.71956 | 0.209667 | NA | 0.744667 | 0.085556 | 0.029556 |
| 4 G | biomass | MET2 | Week 3 | 31 | NA | 79.91756 | 0.206444 | NA | 0.427111 | 0.096444 | 0.033778 |
| 5 L | UA Expt2 | MET2 | 24 | 5 | 0.0146 | 62.0242 | 0.2058 | NA | 0.4651 | 0.21272 | 0.0381 |
| 6 R | UA Expt2 | MET2 | 24 | 5 | 0 | 75.2742 | 0.2002 | NA | 0.3565 | 0.1088 | 0.0143 |
| 7 S | UA Expt2 | MET2 | 24 | 5 | 0 | 72.6162 | 0.2073 | NA | 0.3385 | 0.1116 | 0.0171 |
| 8 T | UA Expt2 | MET2 | 24 | 5 | 0 | 70.1 [symbol] | 0.2554 | NA | 0.4034 | 0.135 | 0.0097 |
| 9 U | UA Expt2 | MET2 | 24 | 5 | 0 | 76.5915 | 0.2335 | NA | 0.4284 | 0.1443 | 0.0148 |
| 10 V | UA Expt2 | MET2 | 24 | 5 | 0 | 68.7213 | 0.1977 | NA | 0.4058 | 0.1235 | 0.0122 |
| | | MET 2 | Week 3 | [symbol] | 0.002435 | 70.90188 | 0.21665 | NA | 0.3898 | 0. [symbol] | 0.0177 |

| | | | | | | | | | | | |
|---|---|---|---|---|---|---|---|---|---|---|---|
| id | expt | donor | day | run | beta [symbol] | Betaine | Bu [symbol] | Choline | [symbol] | F [symbol] | Fr [symbol] |

Two runs of ecosystem MET-1

| | | | | | | | | | | | |
|---|---|---|---|---|---|---|---|---|---|---|---|
| 1 AF | RP1 | MET1 | Week 2 | [symbol] | 0.0 [symbol] | 0.2151 | [symbol] | 0. [symbol] | 15. [symbol] | 0.1132 | 0.4274 |
| 2 B | [symbol] | MET1 | Week 2 | 1 | 0.023778 | 0.279556 | 16. [symbol] | [symbol] | [symbol] | [symbol] | [symbol] |

Three runs of ecosystem MET2

| | | | | | | | | | | | |
|---|---|---|---|---|---|---|---|---|---|---|---|
| 3 C | biomass | MET2 | Week 3 | 32 | NA | 0.005333 | 15. [symbol] | 0.1 [symbol] | [symbol] | NA | [symbol] |
| 4 G | biomass | MET2 | Week 3 | 31 | NA | 0.008222 | [symbol] | 0.1 [symbol] | [symbol] | NA | [symbol] |
| 5 [symbol] | UA Expt2 | MET2 | 24 | 5 | 0.0 [symbol] | 0 | 1. [symbol] | 0. [symbol] | [symbol] | [symbol] | NA |
| 6 [symbol] | UA Expt2 | MET2 | 24 | 5 | 0.0 [symbol] | 0 | 11. [symbol] | 0. [symbol] | [symbol] | [symbol] | NA |
| 7 [symbol] | UA Expt2 | MET2 | 24 | 5 | 0.0 [symbol] | 0 | 12. [symbol] | 0. [symbol] | [symbol] | 0.0 [symbol] | NA |
| 8 T | UA Expt2 | MET2 | 24 | 5 | 0.0 [symbol] | 0 | 12. [symbol] | 0. [symbol] | [symbol] | 0.0 [symbol] | NA |
| 9 U | UA Expt2 | MET2 | 24 | 5 | 0.0 [symbol] | 0 | 12. [symbol] | 0. [symbol] | [symbol] | 0. [symbol] | NA |
| 10 V | UA Expt2 | MET2 | 24 | 5 | 0.0 [symbol] | 0 | 11. [symbol] | 0. [symbol] | 14. [symbol] | 0.0 [symbol] | NA |
| | | MET 2 | Week 3 | 5 | 0.0 [symbol] | 0 | 12. [symbol] | 0. [symbol] | 14. [symbol] | 0.0 [symbol] | [symbol] |

| | | | | | | | | | | | |
|---|---|---|---|---|---|---|---|---|---|---|---|
| id | expt | donor | day | run | Fumarate | Gal [symbol] | Glut [symbol] | Glycine | Glycolate | Histidine | Hyd [symbol] |

Two runs of ecosystem MET-1

| | | | | | | | | | | | |
|---|---|---|---|---|---|---|---|---|---|---|---|
| 1 AF | RP1 | MET1 | Week 2 | [symbol] | 0.0 [symbol] | NA | 0 | 1.022 [symbol] | 0.29 [symbol] | NA | NA |
| 2 B | [symbol] | MET1 | Week 2 | 1 | 0.023778 | 0.441667 | 0.542 [symbol] | 1. [symbol] | 0.2 [symbol] | 0.0 [symbol] | 0. [symbol] |

Three runs of ecosystem MET2

| | | | | | | | | | | | |
|---|---|---|---|---|---|---|---|---|---|---|---|
| 3 C | biomass | MET2 | Week 3 | 32 | 0.0 [symbol] | 0.134 | 2. [symbol] | 0.0 [symbol] | 0. [symbol] | NA | 0. [symbol] |
| 4 [symbol] | biomass | MET2 | Week 3 | 31 | 0. [symbol] | 0.092444 | 2.3 [symbol] | [symbol] | 0. [symbol] | NA | 0. [symbol] |
| 5 [symbol] | UA Expt2 | MET2 | 24 | 5 | 0.0 [symbol] | NA | 1. [symbol] | 0.2 [symbol] | 0.2 [symbol] | NA | NA |
| 6 R | UA Expt2 | MET2 | 24 | 5 | 0.0 [symbol] | NA | 1. [symbol] | 0. [symbol] | 0. [symbol] | NA | NA |
| 7 [symbol] | UA Expt2 | MET2 | 24 | 5 | 0.0 [symbol] | NA | 1. [symbol] | 0. [symbol] | 0. [symbol] | NA | NA |
| 8 T | UA Expt2 | MET2 | 24 | 5 | 0.0 [symbol] | NA | 1. [symbol] | 0. [symbol] | 0. [symbol] | NA | NA |
| 9 [symbol] | UA Expt2 | MET2 | 24 | 5 | 0.0 [symbol] | NA | 1. [symbol] | 0. [symbol] | 0. [symbol] | NA | NA |
| 10 [symbol] | MET2 | MET2 | 24 | 5 | 0.0 [symbol] | NA | 1 [symbol] | 0. [symbol] | 0. [symbol] | NA | NA |
| | MET 2 | Week 3 | Week 3 | 5 | 0.0 [symbol] | [symbol] | 1. [symbol] | 0.1 [symbol] | 0. [symbol] | [symbol] | [symbol] |

| | | | | | | | | | | | |
|---|---|---|---|---|---|---|---|---|---|---|---|
| id | expt | donor | day | run | [symbol] | [symbol] | [symbol] | Lactate | Leucine | Lysine | Methanol |

Two runs of ecosystem MET-1

| | | | | | | | | | | | |
|---|---|---|---|---|---|---|---|---|---|---|---|
| 1 AF | RP1 | MET1 | Week 2 | [symbol] | 0. [symbol] | 0. [symbol] | 0. [symbol] | 0. [symbol] | 0. [symbol] | 0. [symbol] | 1. [symbol] |
| 2 B | [symbol] | MET1 | Week 2 | 1 | 0. [symbol] | 0. [symbol] | 0. [symbol] | 0. [symbol] | 0. [symbol] | 0. [symbol] | 0. [symbol] |

Three runs of ecosystem MET2

| | | | | | | | | | | | |
|---|---|---|---|---|---|---|---|---|---|---|---|
| 3 C | biomass | MET2 | Week 3 | 32 | 0. [symbol] | 0. [symbol] | 0. [symbol] | NA | 0. [symbol] | 0 | 4.080444 |
| 4 G | biomass | MET2 | Week 3 | 31 | 0. [symbol] | 0. [symbol] | 0. [symbol] | NA | 0. [symbol] | 0 | 4.081111 |
| 5 L | UA Expt2 | MET2 | 24 | 5 | 0. [symbol] | 0. [symbol] | 0. [symbol] | 0. [symbol] | 0. [symbol] | 0. [symbol] | [symbol] |
| 6 [symbol] | UA Expt2 | MET2 | 24 | 5 | 0. [symbol] | 0. [symbol] | 0. [symbol] | 0. [symbol] | 0. [symbol] | 0. [symbol] | [symbol] |
| 7 [symbol] | UA Expt2 | MET2 | 24 | 5 | 0. [symbol] | 0. [symbol] | 0. [symbol] | 0. [symbol] | 0. [symbol] | 0. [symbol] | [symbol] |
| 8 T | UA Expt2 | MET2 | 24 | 5 | 0. [symbol] | 0. [symbol] | 0. [symbol] | 0. [symbol] | 0. [symbol] | 0. [symbol] | [symbol] |
| 9 U | UA Expt2 | MET2 | 24 | 5 | 0. [symbol] | 0. [symbol] | 0. [symbol] | 0. [symbol] | 0. [symbol] | 0. [symbol] | [symbol] |
| 10 V | UA Expt2 | MET2 | 24 | 5 | 0. [symbol] | 0. [symbol] | 0. [symbol] | 0. [symbol] | 0. [symbol] | 0. [symbol] | [symbol] |
| | | MET 2 | Week 3 | 5 | 0. [symbol] | 0. [symbol] | 0. [symbol] | 0. [symbol] | 0. [symbol] | 0. [symbol] | [symbol] |

| | | | | | | | | | | | |
|---|---|---|---|---|---|---|---|---|---|---|---|
| id | expt | donor | day | run | M [symbol] | P [symbol] | Phe [symbol] | Proline | Pro [symbol] | Py [symbol] | Serine |

Two runs of ecosystem MET-1

| | | | | | | | | | | | |
|---|---|---|---|---|---|---|---|---|---|---|---|
| 1 AF | RP1 | MET1 | Week 2 | [symbol] | 0. [symbol] | 0. [symbol] | 0. [symbol] | NA | [symbol] | 0. [symbol] | NA |
| 2 B | [symbol] | MET1 | Week 2 | 1 | 0. [symbol] | [symbol] | 0.1 [symbol] | 0. [symbol] | 19. [symbol] | 0.0 [symbol] | 0. [symbol] |

Three runs of ecosystem MET2

| | | | | | | | | | | | |
|---|---|---|---|---|---|---|---|---|---|---|---|
| 3 [symbol] | biomass | MET2 | Week 3 | 31 | 0. [symbol] | 0. [symbol] | 0. [symbol] | 0. [symbol] | [symbol] | NA | 0. [symbol] |
| 4 [symbol] | biomass | MET2 | Week 3 | 32 | 0. [symbol] | 0. [symbol] | 0. [symbol] | 0. [symbol] | [symbol] | NA | 0. [symbol] |
| 5 L | UA Expt2 | MET2 | 24 | 5 | 0. [symbol] | 0. [symbol] | 0. [symbol] | 0. [symbol] | [symbol] | 0. [symbol] | 0. [symbol] |
| 6 R | UA Expt2 | MET2 | 24 | 5 | 0. [symbol] | 0. [symbol] | 0. [symbol] | 0. [symbol] | [symbol] | 0. [symbol] | 0. [symbol] |
| 7 S | UA Expt2 | MET2 | 24 | 5 | 0. [symbol] | 0. [symbol] | 0. [symbol] | 0. [symbol] | [symbol] | 0. [symbol] | 0. [symbol] |
| 8 T | UA Expt2 | MET2 | 24 | 5 | 0. [symbol] | 0. [symbol] | 0. [symbol] | 0. [symbol] | [symbol] | 0. [symbol] | 0. [symbol] |
| 9 U | UA Expt2 | MET2 | 24 | 5 | 0. [symbol] | 0. [symbol] | 0. [symbol] | 0. [symbol] | [symbol] | 0. [symbol] | 0. [symbol] |
| 10 V | UA Expt2 | MET2 | 24 | 5 | 0. [symbol] | 0. [symbol] | 0. [symbol] | 0. [symbol] | [symbol] | 0. [symbol] | 0. [symbol] |
| | | MET 2 | Week 3 | 5 | 0. [symbol] | 0. [symbol] | 0. [symbol] | 0. [symbol] | [symbol] | 0. [symbol] | 0. [symbol] |

| | | | | | | | | | | | |
|---|---|---|---|---|---|---|---|---|---|---|---|
| id | expt | donor | day | run | Succinate | Threonine | Thymine | Trimethyl [symbol] | Tyrosine | Urac [symbol] | Valerate |

Two runs of ecosystem MET-1

| | | | | | | | | | | | |
|---|---|---|---|---|---|---|---|---|---|---|---|
| 1 AF | RP1 | MET1 | Week 2 | [symbol] | 7. [symbol] | NA | 0. [symbol] | 0. [symbol] | 0. [symbol] | 0. [symbol] | 1. [symbol] |
| 2 B | [symbol] | MET1 | Week 2 | 1 | 20. [symbol] | 0. [symbol] | 0. [symbol] | 0.054 | 0.174 | 0.04 [symbol] | 1. [symbol] |

Three runs of ecosystem MET2

| | | | | | | | | | | | |
|---|---|---|---|---|---|---|---|---|---|---|---|
| 3 [symbol] | biomass | MET2 | Week 3 | 32 | 0 | 0. [symbol] | 0. [symbol] | 0. [symbol] | 0. [symbol] | 0. [symbol] | 1. [symbol] |
| 4 [symbol] | biomass | MET2 | Week 3 | 31 | 0 | 0. [symbol] | 0. [symbol] | 0. [symbol] | 0. [symbol] | 0. [symbol] | 0. [symbol] |
| 5 L | UA Expt2 | MET2 | 24 | 5 | 0.0168 | 0. [symbol] | 0. [symbol] | 0. [symbol] | 0. [symbol] | 0. [symbol] | 0. [symbol] |
| 6 R | UA Expt2 | MET2 | 24 | 5 | 0 | 0. [symbol] | 0. [symbol] | 0. [symbol] | 0. [symbol] | 0. [symbol] | 0. [symbol] |
| 7 S | UA Expt2 | MET2 | 24 | 5 | 0 | 0. [symbol] | 0. [symbol] | 0. [symbol] | 0. [symbol] | 0. [symbol] | 0. [symbol] |
| 8 T | UA Expt2 | MET2 | 24 | 5 | 0.0183 | 0. [symbol] | 0. [symbol] | 0. [symbol] | 0. [symbol] | 0. [symbol] | 0. [symbol] |
| 9 U | UA Expt2 | MET2 | 24 | 5 | 0.0114 | 0. [symbol] | 0. [symbol] | 0. [symbol] | 0. [symbol] | 0. [symbol] | 0. [symbol] |
| 10 V | UA Expt2 | MET2 | 24 | 5 | 0.0 [symbol] | 0. [symbol] | 0. [symbol] | 0. [symbol] | 0. [symbol] | 0. [symbol] | 0. [symbol] |
| | | MET 2 | Week 3 | [symbol] | | 0. [symbol] | 0. [symbol] | 0. [symbol] | 0. [symbol] | 0. [symbol] | 0. [symbol] |

| | | | | | | | | | | | |
|---|---|---|---|---|---|---|---|---|---|---|---|
| id | expt | donor | day | run | Valine | Glucose | Glycerol | Methyl [symbol] | Py [symbol] | Xy [symbol] | [symbol] |

Two runs of ecosystem MET-1

| | | | | | | | | | | | |
|---|---|---|---|---|---|---|---|---|---|---|---|
| 1 AF | RP1 | MET [symbol] | Week 2 | [symbol] | 0. [symbol] | NA | 0 | 0. [symbol] | 0 | NA | NA |
| 2 B | [symbol] | MET [symbol] | Week 2 | 1 | 0. [symbol] | NA | NA | NA | NA | NA | NA |

Three runs of ecosystem MET2

| | | | | | | | | | | | |
|---|---|---|---|---|---|---|---|---|---|---|---|
| 3 [symbol] | biomass | MET2 | Week 3 | 32 | 0. [symbol] | 0. [symbol] | [symbol] | [symbol] | [symbol] | 0. [symbol] | NA |
| 4 [symbol] | biomass | MET2 | Week 3 | 31 | 0. [symbol] | 0. [symbol] | [symbol] | 0.0 [symbol] | [symbol] | 0. [symbol] | NA |
| 5 L | UA Expt2 | MET2 | 24 | 5 | 0. [symbol] | 0. [symbol] | [symbol] | 0. [symbol] | 0. [symbol] | NA | 0.0 [symbol] |
| 6 R | UA Expt2 | MET2 | 24 | 5 | 0. [symbol] | 0. [symbol] | [symbol] | 0. [symbol] | 0. [symbol] | NA | 0 |
| 7 S | UA Expt2 | MET2 | 24 | 5 | 0. [symbol] | 0. [symbol] | NA | 0. [symbol] | 0. [symbol] | NA | 0 |
| 8 T | UA Expt2 | MET2 | 24 | 5 | 0. [symbol] | 0. [symbol] | NA | 0. [symbol] | 0. [symbol] | NA | 0 |
| 9 U | UA Expt2 | MET2 | 24 | 5 | 0. [symbol] | 0. [symbol] | NA | 0. [symbol] | 0. [symbol] | NA | 0 |
| 10 V | UA Expt2 | MET2 | 24 | 5 | 0. [symbol] | 0. [symbol] | NA | 0. [symbol] | 0. [symbol] | NA | 0 |
| | | MET 2 | Week 3 | 5 | 0. [symbol] | 0. [symbol] | [symbol] | 0. [symbol] | 0. [symbol] | NA | 0.0 [symbol] |

| | | | | | | | | | | | |
|---|---|---|---|---|---|---|---|---|---|---|---|
| id | expt | donor | day | run | Acetoacete | Acetone | Diethy [symbol] | [symbol] | Histamine | Iso [symbol] | [symbol] |

Two runs of ecosystem MET-1

| | | | | | | | | | | | |
|---|---|---|---|---|---|---|---|---|---|---|---|
| 1 AF | RP1 | MET1 | Week 2 | [symbol] | NA | [symbol] | NA | [symbol] | [symbol] | [symbol] | [symbol] |
| 2 B | [symbol] | MET2 | Week 2 | 1 | NA | NA | NA | NA | NA | NA | NA |

Three runs of ecosystem MET2

| | | | | | | | | | | | |
|---|---|---|---|---|---|---|---|---|---|---|---|
| 3 [symbol] | biomass | MET2 | Week 3 | 32 | NA | NA | NA | NA | NA | NA | NA |
| 4 [symbol] | biomass | MET2 | Week 3 | 31 | NA | NA | NA | NA | NA | NA | NA |
| 5 L | UA Expt2 | MET2 | 24 | 5 | 0. [symbol] | NA | 0. [symbol] | NA | 0. [symbol] | 0. [symbol] | 0. [symbol] |
| 6 R | UA Expt2 | MET2 | 24 | 5 | 0. [symbol] | NA | 0. [symbol] | NA | 0. [symbol] | 0. [symbol] | 0. [symbol] |
| 7 S | UA Expt2 | MET2 | 24 | 5 | 0. [symbol] | NA | 0. [symbol] | NA | 0. [symbol] | 0. [symbol] | 0. [symbol] |
| 8 T | UA Expt2 | MET2 | 24 | 5 | 0. [symbol] | NA | 0. [symbol] | NA | 0. [symbol] | 0. [symbol] | 0. [symbol] |
| 9 U | UA Expt2 | MET2 | 24 | 5 | 0. [symbol] | NA | 0. [symbol] | NA | 0. [symbol] | 0. [symbol] | 0. [symbol] |
| 10 V | UA Expt2 | MET2 | 24 | 5 | 0. [symbol] | NA | 0. [symbol] | NA | 0. [symbol] | 0. [symbol] | 0. [symbol] |
| | | MET 2 | Week 3 | 5 | 0. [symbol] | [symbol] | 0. [symbol] | [symbol] | 0. [symbol] | 0. [symbol] | 0. [symbol] |

| | | | | | | | | | | |
|---|---|---|---|---|---|---|---|---|---|---|
| id | expt | donor | day | run | N,N-Dim [symbol] | S [symbol] | Tar [symbol] | Ty [symbol] | [symbol] | Xa [symbol] |

Two runs of ecosystem MET-1

| | | | | | | | | | | |
|---|---|---|---|---|---|---|---|---|---|---|
| 1 AF | RP1 | MET1 | Week 2 | 6 | 0.004 | 0.00 [symbol] | NA | 0. [symbol] | 0. [symbol] | 0. [symbol] |
| 2 B | [symbol] | MET1 | Week 2 | 1 | NA | NA | NA | NA | NA | NA |

Three runs of ecosystem MET2

| | | | | | | | | | | |
|---|---|---|---|---|---|---|---|---|---|---|
| 3 [symbol] | biomass | MET2 | Week 3 | 32 | NA | NA | NA | NA | NA | NA |
| 4 [symbol] | biomass | MET2 | Week 3 | 31 | NA | NA | NA | NA | NA | NA |
| 5 L | UA Expt2 | MET2 | 24 | 5 | NA | 0. [symbol] | 0. [symbol] | 0. [symbol] | 0. [symbol] | 0. [symbol] |
| 6 R | UA Expt2 | MET2 | 24 | 5 | NA | 0. [symbol] | 0. [symbol] | 0. [symbol] | 1 [symbol] | 0. [symbol] |
| 7 S | UA Expt2 | MET2 | 24 | 5 | NA | 0. [symbol] | 0. [symbol] | 0. [symbol] | 1. [symbol] | 0. [symbol] |
| 8 T | UA Expt2 | MET2 | 24 | 5 | NA | 0. [symbol] | 0. [symbol] | 0. [symbol] | 0. [symbol] | 0. [symbol] |
| 9 U | UA Expt2 | MET2 | 24 | 5 | NA | 0. [symbol] | 0. [symbol] | 0. [symbol] | 1. [symbol] | 0. [symbol] |
| 10 V | UA Expt2 | MET2 | 24 | 5 | NA | 0. [symbol] | 0. [symbol] | 0. [symbol] | 1. [symbol] | 0. [symbol] |
| | | MET 2 | Week 3 | 5 | na | 0. [symbol] | 0. [symbol] | 0. [symbol] | 1. [symbol] | 0. [symbol] |

[symbol]

indicates data missing or illegible when filed

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method for characterizing a first composition comprising a fecal-derived bacterial population, comprising the steps of:
a. obtaining a live culture of the first composition comprising a first fecal-derived bacterial population, wherein the first composition comprising a first fecal-derived bacterial population comprises at least one bacterial strain at a level below a threshold for detection;
b. supplying factors that selectively expand the at least one bacterial strain at a level below a threshold for detection above the threshold level for detection by seeding a chemostat containing culture medium with a second composition comprising a second fecal-derived bacterial population;
c. adding the live culture of the first composition comprising the first fecal-derived bacterial population to the seeded chemostat, and culturing the first composition comprising the first fecal-derived bacterial population with the culture medium from the second composition comprising the second fecal-derived bacterial population in the chemostat for a time sufficient to expand the at least one bacterial strain at a level below a threshold for detection above the threshold level for detection;
d. removing a sample of the chemostat culture after the sufficient time; and
e. identifying the at least one bacterial strain at a level below a threshold for detection.

2-3. (canceled)

4. The method of claim 1, wherein the at least one bacterial strain at a level below a threshold for detection is identified by comparing a bacterial 16S rRNA profile of the first composition comprising a fecal-derived bacterial population obtained prior to culture in the chemostat, to a bacterial 16S rRNA of the chemostat culture medium, obtained after culture for a sufficient time.

5. The method of claim 1, wherein the first composition comprising a fecal-derived bacterial population comprises an ecosystem of a healthy patient.

6. The method of claim 1, wherein the second composition comprising a fecal-derived bacterial population comprises an ecosystem of a healthy patient.

7. The method of claim 1, wherein the first and the second composition comprising a fecal-derived bacterial population are the same.

8. The method of claim 1, wherein the first composition comprising a fecal-derived bacterial population is derived from a patient with a gut dysbiosis.

9. The method of claim 1, wherein the first composition comprising a fecal-derived bacterial population is derived from a patient with a gastrointestinal disease.

10-11. (canceled)

12. The method of claim 1, wherein the gastrointestinal disease is selected from the group consisting of: dysbiosis, Clostridium difficile (Clostridioides difficile) infection, Crohn's disease, ulcerative colitis, irritable bowel syndrome, inflammatory bowel disease and diverticular disease.

13. The method of claim 1, wherein the at least one bacterial strain above the threshold level for detection is identified using PCR on a sample of the first culture, using primers specific for the newly identified bacterial strain.

14. The method of claim 1, further comprising isolating and purifying the at least one bacterial strain above the threshold for detection.

15. The method of claim 1, wherein the factors comprise at least one of butyrate, propionate, acetate, mucin, vitamins, or antibiotics.

16. The method of claim 1, wherein additional factors are added to the live culture or the culture medium from the second composition, wherein the additional factors comprise at least one of butyrate, propionate, acetate, mucin, vitamins, or antibiotics.
